SEQUENCE LISTING (102 sequences)

SEQ ID NO: 1 - protein, 217 aa, Borrelia burgdorferi
MQSGLKIKLILFFCCFACSCDINYPEIKELDYKINYYFTENRLDYSMSFDFAIKVINSKDVFKLSIENKNTNEFIQVINNNYSSFFIDSSLGKDILYCKDLRFNFFDKTFEDFTSCVRLFDKGMRVYNRELVISLGMSKYDLDDVHNYVYKSKDMEMLNKLSNSKVFFVKTYKDKLHPVSSVVRIDSIDILEIDKAFDNYISFYYVEKNSNLFFKVG

SEQ ID NO: 2 - protein, 464 aa, Borrelia burgdorferi
MRIKIKDILISSKDVKFVGNIKNIERVVSFYSLDSREIKDDNINDSLYFAYKGNKVDGFSFVKYLIDLGVKCFACSREHESECIKYLNDNEGLVFLLTSNVIKLLQALASFLIEKTSFKRIAITGSNGKTTTKEMLYSILSKKYKTYKTWGNLNSDIGLPLSILRVEGNEEYAVFEVGVSYVGEMDLLSQILKPEIVIITNISYAHMQAFKELQAIAFEKSKIIGKNIEIFVVNEMNDYCVYLEKRAKIANPNVKIVYFDFENLSIKSFSFLDGKFSFDFVYKGFEYSILLLGRHNIFNAIGCINLALFLGMREKEIKEGLIETAFQKGRAEILTKNGYLILNDSYNGNMGSFMALKNMILDLNIQNKKFIVLGSFKELGELAYKTHKDLIQEAISMNFDKIFLIGEEFLDVRDSENLVEKCLYYFSEFDKFIDFFLKSLEPSVFIVIKGSRFNRLERILNIFR

SEQ ID NO: 3 - protein, 475 aa, Borrelia burgdorferi
MIKMSLNYTEINTLIKEIPFTNSLITKIIQPDYKSLVLEIYNKIDNKKFKILICLNPNTTRFHITKKNFKKNALKLRFSDFLKSKIQNGKIIKAFQMKNERIISLEILQKDMIILFIKLWPSSPNIIATNSNFKILDAYYRRPKIKETTGEIFLKAKEIHESNKMSDKKIMELKEEYNNTSYTSYSEFLENYYESLNDQIKKTNIKELLIEKYKKELIVLEKRIDSLKQQIKLLENIENEKEKGELILLNINKIQKGIKEINLLNYKEEKIKISLNQSLSPKENALQYFKAYKKGKNSFKTIQNQLKDNLDKFNLIQSKITMLKVENLIPEEEYNQEKTAIKEKEKTPKIGLHFTYCGFEILIGRNAKENDKLLRHCVKGNDYWLHTRDYPGAYVFIKNQKNKTPSLDVLLGAGNLCVFYTKLAKKSGKADLYYTQVKNLRRVKNKKLGLVIPKAEKNLHIKLDENLIKKIKNQT

SEQ ID NO: 4 - protein, 599 aa, Borrelia burgdorferi
MGVITNFRYKFGIFFLIVIMVLYLAILFQMQIGKHLFYDREANVFLSRLEKINASRGEILDSNSNVLANNLTMFILKISLQQYYNMPAATRIEMIDFLSSTLDIDKSIILSKLQEPGGYLKDVEIIELTPKMLFKISEKKFYYPALLWTYSFKRNYLVDDSYSHSIGYVGQINQRELRTFYNVSGYDNTSTIGKLGVEQVYDNYIRGQEGLIKYKVDSKERRIDDGSIIKNMVPGNDVVLNINKDIQDLAKNALGKRYGSVVVLKPSTGAVLALHNYPYYSMKDVYNKDNKEDYSFLNKAIQSVYPPASIFKLVVAAAILEERVIDKDRKIYCPGYFKVGNRIFHCWKPGGHGYVNLEEAIAHSSNVYFYTLGLKYLGVDRIRKYAKEFGFGEKTGIDLPNEVAGLLPSPEWKEKTFNQPWVGGDTVNFSIGQGFLNATPMQIVNMVAMIANEGVVYKPRIVNKILKGGTNKVVLENKPEILRKTNLISKNTFKLLKKYMRSVITYGTARYAVLTKAVEVGGKTGTGQTGIDGFENSSFIGLAPYNGSADNQIIVFSLVEAKSNVDWWPAKSTDLIMQGIFANQSYEDILKGYRPWYIR

SEQ ID NO: 5 - protein, 295 aa, Borrelia burgdorferi
  misc_feature (265)..(265): Xaa can be any naturally occurring amino acid
MSKIFLLFNAGFFFLKIIYVFSYPEIKNFSRQDPVFSDLKIKVLKYNKKQHIPLFFYSYKVKKGDTFFKIANKINGWQSGIATINLLDSPAVSVGQEILIPSKKGVFVFDSKDYRFNNLLLATRDLAKAEKVKIKRNDRVYEFYFFDFVKNPDFGLFSGTELLFFLNANFIFPLKKFIVSSDFGFRNDPFTGNKSFHTGIDLAAPMNAEVYSSSSGIVIEAGYNDLYGNFVVVGHKNNIKSLYGHLNLYSVKIGDFVKSGEFLGXVGQTGRATGPHLHFEILKKNIPINPLKFLK

SEQ ID NO: 6 - protein, 123 aa, Borrelia burgdorferi
MNTKTLYLISLILLACNKNNKIPLIQKLDLPKSSILGFSNKMGIIIKDYAFLSKSTKKNSELDYDYAILLRKDEVVKIEKTLEKTERYGIEGNWILVNYKGTKRYIFSKDINIVNNLIIDHSK

SEQ ID NO: 7 - protein, 389 aa, Borrelia burgdorferi
MNTKATTPLLLLFLIQSLAFSSEIFEFKYIKGSKFRLEGTDNQKIYFNGHYNSSSNTNIQISSEIKDIKENFASIKAFFRILKRENINEPYLLNEEFEEIFSVNKQGEYTIGANQKRPSVRGIPRFPKTPIKINEKWSYLAEEYIEASKIDKSIKDFVVKFNVNYEYKGKEEHNGKHYHIILSNYESQYNVKNISFYQKVDQKIYFDNEIGNTYKYSDKYIFEINQNNNQHFKMIGNSLGRIVSIELPNDNLIETEVENYIREKKIKAIEVEKNNKGINLSFDIEFYPNSFQILQKEYKKIDLIAKLLEKFKKNNILIEGHTEQFGLEEEMHELSEKRARAIGNYLIKMKVKDKDQILFKGWGSQKPKYPKSSPLKAKNRRVEITILNN

SEQ ID NO: 8 - protein, 372 aa, Borrelia burgdorferi
MYNNKTMGSNKTIIINLNNLEHNLNLIKNKIGEKEIVATLKGDAYGHGLINIFKFLKSKNINYFGLSNIEDAKTLKKIDKNIKILMYIKVDKKEIKNLIKFELVPFVSDFEYLFLIEKECALQKNKIKVHLKIDIGMNRYGIKIDDALEIATYIQNSKFLELEGICSHLPSIENFKTTQKQIEQFLFFLETLKQKNIHPKFVHISNSGHIINYKLNPQFNMVRPGLILYGYCQSLKNKKPALNFKPVLSLFSKVIFIKNVKKGEKISYSGIFQAKEDMKIGIIPIGYFDGIPQNISNDFYFLINNKKCKIRGKVCMNLTIVEIPKDLKVKTGSKVEIVSEKLSIDEMSKFSKRSHYELLCNIGKYEKRKYLD

SEQ ID NO: 9 - protein, 233 aa, Borrelia burgdorferi
MLRENKLKNYSLSDVNTSKIPKNELFSSISHLFGIILSIIGTTILVTISTLKKKDLHVVVFLIYGFSMTLLYVMSTLYHIFPKGSKIKKIFRKFDHISIFILIAGTYTPACAILVPNKSGLIILCIVWSLAIIGIIFKIIFTNSPGWFNGSIFIIMGWIIIFKIKPIYKALSGKGFFWLVFGGIVYTIGGIVYILSKKFNPTINMKMHDVFHILIIIASASHFWLMLKYISNF

SEQ ID NO: 10 - protein, 103 aa, Borrelia burgdorferi
MIYRPEGELVINSIFKVKEDLLHIFKKMKEGDTLIIDLSNVEKIDITFIQILYASNKYAKNRNLFVKIEYPSDEVLSSLIYGGFLVDIEDVDSFDLGLNLVGF

SEQ ID NO: 11 - protein, 103 aa, Borrelia afzelii
MIYRPEGELVINSIFKVKEDLLNIFKKMKEGDTLTIDLSNVEKIDITFIQILYASNKYAKNRNLFVKIEYPSDEVLSSLIYGGFLTDIEDVDSFDLGLNLVGF

SEQ ID NO: 12 - protein, 103 aa, Borrelia garinii
MIYRPEGELVVNSIFKVKEDLLHIFKKMKEGDTLTIDLSNVEKIDITFIQILYASNKYAKNKNLFVKIEYPSDEVLSSLIYGGFLIDIEDVDSFDLGANLVGF

SEQ ID NO: 13 - protein, 341 aa, Borrelia burgdorferi
  misc_feature (217)..(217): Xaa can be any naturally occurring amino acid
MKNLIEVLMLTFNEPLYLFLLVIFPLIIYFNHFLKNRGGKIKFPISLYGNFNSLKLKDYRLNLMYFFTYSFLYLAAMVMVFALAGPSVSKKKMIHLSAGADIVIVLDISPSMGAVEFSSKNRLEFSKELIRGFISQRENDNIGLVAFAKDASIVVPITTDREFFNKKLDDIYIMDLGNGSALGLGISIALSHLKHSEALKRSIVVLTDGVVNSDEIXKDQVINLAQGLNVKIYSIGIGSSEEFSVEFKLRSGKFYQGSFKEVYDPSMLVEISNKTGGLFYSVNDDFSFQFAIQDFSKKENLERKIKIAVDNKDIYKEFLVLAFCLLLVYFIFSKIFLKEIL

SEQ ID NO: 14 - protein, 333 aa, Borrelia afzelii
MLTFNEPLYLFLLVIFPITIYFNHFFKNRGGKIKFPISIYGSLNSLKLKDYRLNLMYFFTYSFLYLAAMVMVFALAGPSVSKKKMIHLSAGADIVIVLDISPSMGAVEFSSKNRLEFSKELIKRFISQRENDNIGLVAFAKDASIVVPITTDRDFFNKKLDDIYIMDLGNGSALGLGISIALSHLKHSEALKRSIVVLTDGVVNSDEIYKDQVINLAQGLNVRIYSIGIGSSEEFSVEFKLRSGKFYQGSLKEVYDPSMLVEISNKTGGLFYSVNDDFSFQFAIQDFSKKENLERKIKIAVDNKDIYKEFLVLALCLLLIYFIFSKIFLKEIL

SEQ ID NO: 15 - protein, 333 aa, Borrelia garinii
MLTFNEPLYLFLLVIFPLIIYFNHFFKNRGGKIKFPISLYGNFNSLKLKDYRLNLIYFFTYSFLYLAAMVMVFALAGPSVSKKKMIHLSAGADIVIVLDISPSMGAVEFSSKNRLEFSKELIRRFISQRENDNIGLVAFAKDASIVVPITTDRDFFNKKLDDIYIMDLGNGSALGLGISIALSHLKHSEALKRSIVVLTDGVVNSDEIYKDQVINLAQGLNVKIYSIGIGSSEEFSVEFKLRSGKFYQGSLKEIYDPSMLVEISHKTGGLFYSVNDDFSFQFAIQDFSKKENLERKIKIAVDNKDIYKEFLVLAFFLLFIYFIFSKIFLKEIL

SEQ ID NO: 16 - protein, 473 aa, Borrelia burgdorferi
MLFLFTKFSFVQAQANQILTEISPLSILSKNGKGSVYLKVSKSSDYILTLDKSSNSDFVFKIYDISNKKYITDKVKRRDFKIRLDKNSLYAIIYVGTKNENIKFSLTDLDFSILSSDSLKAKTSKIEKEDLFFTLKDLPVLNLTAKLKKYVLRIYKSNIYIAYQLENSDDIKVAEFIEDVGWFNLDSSVNRNITNIVNFDFSINSKGNLYIAFVTKSGADFASELIVKKFNSRKWIDISPGHIENFGSLLNISIDLKDRLYLAYLREIRGEYKINLISNMGYGSIWTDVIHAYLSKGDSNVNSSNIGLISEPFLGIFYNYKSNNEIKSEFIVNNENAWVNANIPSVYMANFIKGFFDSNFNQIIMSFVSENRPIVNICPLKSSRWINISPNVEMEGLSADIGLYKNNLFLAFEDNNNVRLIYFKNKNWYFLNKLENFKSNVKSPQIGIYGNQGLVISTLSSNSNELFFTLICQ

SEQ ID NO: 17 - protein, 481 aa, Borrelia afzelii
MKINKTLILLFLFTKLFSIQAQVNQILTEISPLSILSKNGKGSVYLKVSKSSDYILTLDKSSNSDFVFKIYDISNKKYITDKIKSKDFKIRLDQNSLYAIIYVGSKNENIKFSLTDLDFSILSGDFLKAKTSKIEKEDSFFTLKDLPVLNLTSKLKKYVLRIYKSNIYIAYQLENSDDIKVAEFIEDVGWFNLDSSVNGNIINIVNFDFSINSKGNLYIAFVTKSGSDFASELVVKKFNSRKWIDISPGYIENFGSLLNMSIDLKDRLYLTYLREIEGEYKINLISNMGYGNIWTDVIHSYLSKGNSNVNSSNIGLISEPFLGIFYNYKSNNEIKSEFIVNKENAWVNANIPSVYMANFIKGFFDSNFNQIIMSFVSENRPIVSICPLKSNRWINISPNVKMEGSSIDIGLYKNNLFLAFEDNNDVRLIYFKNKNWYFLNKLEHFKGNVTSPQIGIYGNQGLVVSTLSSNSNELFFTLVCR

SEQ ID NO: 18 - protein, 481 aa, Borrelia garinii
MKINKILILLFLFTKLSSIQVQVNQILTEISPISILSGNGKGSVYLKVSKSSNYILTLDKSSNSDFVFKIYDISNKKYITDKIKRRDFKIRLDENFLYAIIYVGTKNENIKFSLTDLDFSILNSDSLKAKTSKIEKEDLFFTLKDLPVLNLTAKLKKYILRIYKNNIYIAYQLENSDDIKVAEFIKDVGWFNLDSSVNGNIINIVNFDFSINSKGNLYIAFVTKSGSDFASELVVKKFNSRKWIDISPGYIENFGSLLNISIDLKDRLYLTYLREIGGEYKINLISNMGYGNIWTDVIHSYLSKGDSNVDSSNIGLISEPFLGIFYNYKSNNEIKSEFIVNNENAWANANISSVYMANFIKGFFDSNFNQIIMSFLSENRPIVSICPLKSKRWVNISPNVEMKGSSIDVGFYKNNLFLAFEDNNNVRLIYFKNKNWYFLNKLEYLKNNVASPQIGIYGNKGLVISTLSANSNELFFTLICQ

SEQ ID NO: 19 - protein, 81 aa, Borrelia burgdorferi
MRKDIHPKNNLVVFKDGSNGAMFLTKSTLNSKETIKYIDGKEYPLVTVEITSKSHPFYTGQQKFVDAAGRIDKFNKRYKKS

SEQ ID NO: 20 - protein, 81 aa, Borrelia garinii
MRKGVHPKNNLVVFKDGSNGAMFLTRSTLNSKETIKYTDGKEYPLITVEITSRSHPFYTGQQKFVDAAGRIDKFNKRYKKS

SEQ ID NO: 21 - protein, 111 aa, Borrelia burgdorferi
MVRTDAFFTENNINLVKKNPLHFDVNLFSSKSNAKDNDIKTFKDVLINSITDVNKSQLNVSKVTEQAILKPSSIDVHDVVIAMSKANMNLSILKAVVERGVKAYQDIINIR

SEQ ID NO: 22 - protein, 111 aa, Borrelia garinii
MVRIDAFFTENNINLIKKNPLHLDVNFFSSKSSTKNNDAKTFKDVLINTITDINKSQLNVSKVMEQVVLSPSSVDVHDFAIALSKANMNLSILKAVVERGIKAYQDIINIR

SEQ ID NO: 23 - protein, 531 aa, Borrelia burgdorferi
MEREKLINEMVSKMQDHELLGQMFMISYPNQSITNFVLDFISKKNLGGIKIFGWNAKDLKNLTESIHKAQKTSQNNKFKIPLFVATDQEGGLAQHIKLNTSETIGNLGIAASLSPKDSYNTGYYIAQELRQLGINLNFAPIVDIYSHENNFAIGPRTYSDNPKIVSLLSLAFYKGQKQGGVISTAKHFPGHGNTTLDSHINIPIINSNLLEISLNELLPYKILIQENIPVIMTGHLAYPKLTNGENIPASSSIKIIKDILRKKLKYNNIIITDDLLMNAVNYNNESIYNTIERIVRTKSDIFLISLNENIQQNAYNMLLNLMKKDSEIKNNIIESNKRILRIKLMYLKENKNKSDLYPNLNKNEKIYSKEGEKFFEQNTLRSITKVRIEKEISKTKKTLIISPYYKMIVEGKKIFQNTYAYYYNYYPLNGINPQKLDEIKKLINKFEQVIFNLSTPGSLKYLENLKEYKDKISVIVSLTPHHIKKLNWIKNIVIIYGTTPLAFKSGFLTLTKDFDPKGTIPLKNIINKYYP

SEQ ID NO: 24 - protein, 531 aa, Borrelia garinii
MEREKLINEMVNKMQDHELLGQMFMISYPNQSITNFVLDFISKKNLGGIKIFGWNAKNLKNLTESINKAQKTSQNNKFKIPLFVATDQEGGLAQHIKLNTSETIGNLGIAASLSPKDSYNTGYYIAQELSQLGINLNFAPIVDIYSDENNFTIGPRTYSDNPKIVSLFSLAFYKGQKQGGIISTAKHFPGHGNTTLDSHISIPIINSNLLEINSNELLPYKILIQENIPVIMTGHIAYPKLTNGENIPASSSTKIIKDILRKKLKYNNIIITDDLLMNAVKYNNESIYNTIERIVRTKSDIFLISLNENIQKNAYNMLLGLMKKDSEIKNNIIESNKRILRIKLEYLKENKNQSDLYPNFNKNEKIYSKKGEKFFEQSTLRGITKVRIEKEISKTKKTLIISPYYKMIIEGQKIFQNTYAYYYNYYPLNGINPQKLDEIKKLIKKFDQVIFNLSTPGSLKYLENLKEYKDKISVIVSLTPQHIKKLNWIKNIVIIYGTTPLAFKSGLLTLTKDFGPKGNIPLSNITNKYYP

SEQ ID NO: 25 - protein, 61 aa, Borrelia burgdorferi
MAKKSMIIRALRKPKYKTRQNNRCKLCGRPRGYLRDFCMCRICFRKYASEGLIPGVSKSSW

SEQ ID NO: 26 - protein, 61 aa, Borrelia garinii
MAKKSMIIRALRKPKYKTRQNNRCKLCGRPRGYLRDFCMCRICFRKYASQGLIPGVSKSSW

SEQ ID NO: 27 - protein, 348 aa, Borrelia burgdorferi
MTMLPKIAKEIINEYDQKILPNAILLLGEKFSSKKISAIELAKKILNGKNLTNPNLLIFSNLDTVEAKAHLSTNSQKIANKYLEYIKTVIFTKCYFSNEKNLKKIEKNINYINSVYYEKEYNENIKNELIKNIENINKELNHSITVYDVKKIQTWIFSEKEKPKVIYINEIENLSFNVHNSLLKILEEPPSNIYFILAARNKNKIPKTILSRLRVYNFAKLDRSLEIQRFKESFLINKDITIEEYFASFYKEESKKIKKELAKILNIIKEKKSIFNLEEVDFIKDEQSFKIFLNELTINIRKDFLENKIDINQYLKYTEHLKNIYKYRPYNQNKKLIIENLMLNYEEI

SEQ ID NO: 28 - protein, 348 aa, Borrelia garinii
MTILPKIAKDIIKAYDQKILPNAILLLGEKFSSKKTSAIEIAKKILNEKNLTNPNLLIFANLDTIEAKAHLSTNSYKIINKYLEYIKTVIFTKCYFSNEKNLKKIEKNINYINSVYYKKEYNINIKNEFIKNIENINKELNRSITVYDVKKIQTWIFSEKEKPKVIYINEIENLSFNVHNSLLKILEGPPSNIYFILAARNKNKIPKTILSRLRVYNFTKPEKRLGIQIFKESFLTNKDITIEEYFASFYEEESKKIKKELIKILNIIKEKKSIFNLEEIDFIKDDQSFKIFLNELTINIRKDFLENKIDINQYLKYTEHLKNIYKYRPYNQNKKLIIENLMLNYEEI

SEQ ID NO: 29 - protein, 152 aa, Borrelia burgdorferi
MTIYKKIAMSFYSGILSYFIIAPIFGERGFVNYQKLDNNLTLIKNHIEKLKEIQKELKARYINLQVSKSEILKEAKKLGYYPKNSTVIKTNNNKDQYNQGQILTLQKPLSKNQNFYLISIAIGLIYYFLSSCIIQTKKITKINKLASNNSKD

SEQ ID NO: 30 - protein, 152 aa, Borrelia afzelii
MTIYKKIAMSFYSGILSYFIIAPIFGERGFVNYQKLDNNLTLIKNHIEKLKKNQKELKARYINLQVSKSEILKEGNKLGYYPKNSTVIKTNNNKDQYNQGQILTLQKPLSKNQNFYFISIAIGLIYYFLSSCITQTKKNSKSNKLASNNSKD

SEQ ID NO: 31 - protein, 56 aa, Borrelia burgdorferi
MFRFIKDSILELKKVTWPKYNEVVGNGKQVFWLVLFVSIFLGIVDYLMFLVVTYVF

SEQ ID NO: 32 - protein, 56 aa, Borrelia afzelii
MFRFIKDSILELKKVTWPKYNEVVENGKQVFWLVLFVSIFLGIVDYLMFLVVTYVF

SEQ ID NO: 33 - protein, 124 aa, Borrelia burgdorferi
MKSIGCDIIKVERFKNFLENKKKMERFFTHKEIENFKLKGGSIIESLAGKFAAKESLIKALSPLLQYKINYTLKDIEVIKSLKGNAEFCLHNEVEKFAIKMNLKLYLTISHEKEYAIAFVIVEN

SEQ ID NO: 34 - protein, 124 aa, Borrelia afzelii
MKSIGCDIIKVERFKSFLENKKKMERFFTHKEIENFKLKGGSIIESLAGKFAAKESLIKALTPLLQYKIRYSLKDIEVIKSLKGNAKFCLHNEIEKFAIKMNLKLYLTISHEKEYAIAFVMVEN

SEQ ID NO: 35 - protein, 143 aa, Borrelia burgdorferi
MLSKIFVDADSCNFKIIKFLQKFILHNKSKLIVVSNKFFSLEITENVALEVVDNVDSFILNLADRYSLVITRDIFFAKLLLDLGVKVMNDEGRIFDINNINYLYFRSKINFNLKIKIKKYYDEDFNKSRYEKFITNFYSLFFS

SEQ ID NO: 36 - protein, 143 aa, Borrelia afzelii
MLNKIFVDADSCNFKIIKFLQKFILHNKSKLIVVSNKFLSLEKTENVALEVVDNVDAFILNLADRYSLVITRDIFFAKLLLDLEVKVMNDEGRIFNINNINYLYFRSKINFNLKIKIKKYYDGDFNKSRYKKFITNFYSLFFS

SEQ ID NO: 37 - protein, 416 aa, Borrelia burgdorferi
MILRLPEITKIAYLIFKNSTNKIALIGSGISLSLVMIPLIIVYYMSSNIMTSTINKYIESEGFSMQIEYNETNRTHYLKDRLSLLKKKHNYKDLNYFFEKRTYGIIGNNKKQGVLIRAIENKFILENKSIKLIKGTKNLKKDSILISNQIKNKLNLNLNEKIDILIPNTKNDKIMPRIKKFNISGIIETGLKDIDNNLVLISFENENLMSKKFSKSIIGLKTNSNSIKNNEILKQNLETEFQEFQIKTFYELYSNKYNNLDISKKLLIFIMALIIIFASINISSSLSMLIFENKKKIAILKSIGMNNLNIKIIFLLISLTLSTIFCGIGIIIGNYLTLKISYLINFIDNILNFFLKIFGEENSEILNSEYYVSEFQIHLSLSFSLTLLGLYMLINILTTLIPLNIVSNLKEKEILR

SEQ ID NO: 38 - protein, 417 aa, Borrelia afzelii
MMLLKLTEITKIAYIIFKNSTNKIALIGSGISLSLVMIPLIIVYYMSSNIMTSTINKYIESEGFSTQIEYNDTNKTHQLKDRLDSFKKKYNYKDLNYFFEKRTYGIIGNNKKQGVLIRAIENKFILENKSIKLLQGAKNLKKDSILISNQIKNKLNLNLNEKIDILIPNTKNNKIMPRIKKFNISGIIETGLKDIDNNLVLISFENENLMSKKFSKSIIGVETSSNYIKNNEILKQNLETEFKEFQIKTFYELYLNKYNNLDISKKLLIFIMALIIIFASINISSSLSMLIFENKKKIAILKSIGMNNLNIKIIFLLISLTLSTTFCGIGIIIGNYLTLKISYLINFVDNVLNFFLKIFGEEYSEILNSEYYVSEFQIHLSLSFSLTLLGLYMLISILTTMIPLNIISNLKEKEILR

SEQ ID NO: 39 - protein, 173 aa, Borrelia burgdorferi
MKLPKLYKLILLFLFTTRLFSVKDEKSDNKLELFSNVETKIKKNSKNYDSNSNSKKIKKESILKRDTNSEKNINSNIYIQKSKKINYPNRNLGNNINQKTANDVNFTKTSYVKVYPNYKDDNFQEIKNANKFPAKTEKTHMLIGPILKDNLGIIIKMLKTKGYTLIEYIEDNN

SEQ ID NO: 40 - protein, 173 aa, Borrelia afzelii
MRLTKLYKLILLFLFVIRLFPVKDGKLNNKLELFSNVETKIKKNSKIHDSNSNFKKTKKESILKKNTNVEKNVNSNIYIQKSKKINYPNKNLSNNTNQKTANNGNYTTNHYVKVYPSYKNDNFEAIKNINKFPVKAEKTHALIGPILKDNLGIIIKILKTKGYSLIEYIEDNN

SEQ ID NO: 41 - protein, 419 aa, Borrelia burgdorferi
MIKQKLKISQNLNSIQIQTIKILSLNQKELTKLILEESENNECLEINSNKIFFETLKTYRFKKVFYKEDDMIKNQHDIALEKTQTNTSLKEHLLLQLRIQRINEDEIKIGEILINNLNSKGFHIINPYDLFKKEEKEKVKKIIELIQKFDPIGICVPNIIESLILQAKHHKLETNIIKILEKAELLEKTQKKLKEELKIRSKEFNTALEIIRQKLNPNPTLEFKDPNDTNFYVDPDILIINHNNKFKIKIKEVNIFKKELKRTSENPQKQKKAKWLIESLRYRDEILAKIGIAIYTLQKEFLRRGFKSLRPMNLSILSEKISVSKSTISRAIKNKYLKCEWGTILIKELFSSVGGAKTNEFSKLSIKITVKKLLEANKKMSDKEISVILKSKGISISRRTVNKYRNELKSEKGRTYYGT

SEQ ID NO: 42 - protein, 419 aa, Borrelia afzelii
MIKQKLKLSQNLNSIQIQTIKLLSLNKKELTQLILEEIENNECLKIDSNKIFFETLKTYRFKKVFYKEDNMIKNQHDIALEKTQTNISLKEHLLLQLRIQRINEDELKIGEILINNLNNKGFHIINPYDLFKKEEKEKVKKIIELIQKFDPIGICVPNIIESLILQAKHHKLESNIIKILEKAELLEKTQEKLKEELKIRSKEFNTALEIIRQKLNPNPTFEFKDPEDTNFYVDPDILIINHNNKFKIKIKEVNIFKKELKRTNENPQKQKKAKWLIESLRYRDEILAKIGIAIYTLQKEFLRRGFKSLRPMNLSILSEKINVSKSTISRAIKNKYLKCEWGTILIKELFSSVGGAKTNEFSKLSIKITVKKLLEVNKKMSDKAISAILKSKGISISRRTVNKYRNELKSEKGRIYYGT

SEQ ID NO: 43 - protein, 262 aa, Borrelia burgdorferi
MNKPLLSELIIDIGNTSIAFALFKDNQVNLFIKMKTNLMLRYDEVYSFFEENFDFNVNKVFISSVVPILNETFKNVIFSFFKIKPLFIGFDLNYDLTFNPYKSDKFLLGSDVFANLVAAIENYSFENVLVVDLGTACTIFAVSRQDGILGGIINSGPLINFNSLLDNAYLIKKFPISTPNNLLERTTSGSVNSGLFYQYKYLIEGVYRDIKQMYKKKFNLIITGGNADLILSLIEIEFIFNIHLTVEGVRILGNSIDFKFVN

SEQ ID NO: 44 - protein, 258 aa, Borrelia afzelii
MNKPLLSELIIDIGNTSIAFASFKDNKINLFVKMKTNLMLRHDEVFSFFKENFDFNVNQVFISSVVPILNGIFENVIFSFFKIKPLFISFDLNYDLTFNPYKSGKFLLGSDVFANLVAAIENYSFENVLVVDLGTACTIFAVSRQDGILGGLINSGPLINFNSLLDNAYLLKKFSISTPSNLLERTTSGSVNSGLFYQYKYLIEGVYRDIKKMYKKEFNLIIAGGNANLLLPLIEIEFIFNIHLTVEGIRILGNSIVF

SEQ ID NO: 45 - protein, 239 aa, Borrelia burgdorferi
MKFTVLSLFPAIIKPFFENSIMKKAINKGIVSFELVDVRDFSKDKHKRCDDLPYGGGAGMVLKAEPISFALEHVESAKKTTIFLSPSGIKYSQELAYSLSKREEIVIICGRYEGIDQRIIDLYVDFEISIGDYVLSSGEIAALVLIDSVYRLLDGVINPNSLLEESFGVKNGLLEYPHYTRPYDFKGIKVPEVLLSGHHANIKNWRLVKAREKTKKNRYDLYLKYLEIIGEDNGFDKKN

SEQ ID NO: 46 - protein, 239 aa, Borrelia afzelii
MKFTVISLFPVIIKPFFENSIMKKAINKGIVSFELVDVRDFSKDKHKRCDDLPYGGGAGMVLKAEPMFFALEHVEAAKKTTIFLSPSGIKYTQELAYSLSKKEEIVIICGRYEGIDQRIIDLYVDFEISIGDYVLSSGEISALVLIDSIYRLLDGVINPNSLLEESFGMKNGLLEYPHYTRPYNFMGIKVPEVLVSGHHENIKNWRLLKAIEKTKKNRYDLYLKYLEIIGEDNGFDKKN

SEQ ID NO: 47 - protein, 86 aa, Borrelia afzelii
MQLISLNNIEKQESFIYYRNVYFADVIYKYNDVLEIKKVKFVIEVTPLGEKHVTIDFIDFLNYPVLKLMVAIKRRIIDLEFKGKLP

SEQ ID NO: 48 - protein, 86 aa, Borrelia garinii
MQLISLNNIEKQESYIYYRNVYFADVVYKYNDTLEVKKVKFVIEATPLGEKHVTIDFVDLLNYPVLKLMVAIKRCVIDLEFKGMLP

SEQ ID NO: 49 - protein, 314 aa, Borrelia afzelii
MYKIQKTLLILCILGIENINSEIINTIPKVLYKKEAFQGDYIYFASNKNFKRLSLLSINKNPIISSSPFKFTVGSKTYYIAFIGITPMIKEGKRKIQIEYKNKSYIKEIEIKKFNFKKTKISFNKEKAKLITQKKSIKQKEQALVLWNIIGNIGDTTIYHYDTLVKPIKDQYIITSQYGDLRLYMQGSKKISNYTMHNGIDYAPFKREKTPIFAAGKGKVVFAQNRELTGNTLIIQHLPGIFTIYLHLSKLGTSENKVVNAGEYIGHTGNTGLSTGPHLHFEARINGIAINPDFLLNGMLIDKNKIINNIKRIE

SEQ ID NO: 50 - protein, 314 aa, Borrelia garinii
MYRIQKTLLIFCILGIENINSEIINTIPKVQYKKEAFQGDYIYFASNENFKKLSLLSINKNPIISSSPFKFTVGSKTYYIAFIGITPMIKEGKRKIQIEFKNKSYVKEVEIKKFNFKKTTISFNKEKAKLITQKKSIKQKEQALVLWNIIGNIGDTTIYHYDALVKPIKDQYIITSQYGDLRLYMQGSKKISNYTMHNGIDYAPFKRENTPIFAAGKGKVVFAQNRELTGNTLIIQHLPGVFTIYLHLSKLGISENKVVSAGEYVGHTGNTGLSTGPHLHFEVRINGIAINPDFLLNGMLIDKNKIINNIKRIE

SEQ ID NO: 51 - protein, 254 aa, Borrelia afzelii
MRKCFNFFLFFGVTSVSFAQTKSLQATNGLNFPFLNFENIGGSEIAFSLQLLILLTIITLSPAFLVLMTSFLRISIVLDFIRRALSLQQSPPTQIVMGLALFLTIFTMWPTFNVIYKQAYLPLKESKINFDEFYNKGIAPLRIFMYKQMSDGRHEEIRLFMSISSYDRPKNFSEVPTHVLIAAFILHELKVAFKMGILIFLPFIVLDIIVASVLMAMGMIMLPPVMISLPLKLILFVMVDGWTLITSGLIKSFM

SEQ ID NO: 52 - protein, 254 aa, Borrelia garinii
MRKCFNFFLFFSVTSLSFAQTKSLQAAKGLNFPFLNFENIGGSEIAFSLQLLILLTIITLSPAFLVLMTSFLRISIVLDFIRRALSLQQSPPTQIVMGLALFLTIFTMWPTFNVIYKQAYLPLKESKINFDEFYNKGIAPLRIFMYKQMSDGRHEEIRLFMSISNYDRPKNFSEVPTHVLIAAFILHELKVAFKMGILIFLPFIVLDIIVASVLMAMGMIMLPPVMISLPFKLILFVMVDGWTLITSGLIKSFM

SEQ ID NO: 53 - protein, 99 aa, Borrelia afzelii
MAVNPLDFLKNMSSVKSNIDNIKKEMSKITVCGKAGSNIVTIEMDGEFNVKKVSINKEFFDDLDNDAFEQMVKSALNDAVSKVKEEIKLKTMGVLPFGM

SEQ ID NO: 54 - protein, 99 aa, Borrelia garinii
MAVNPLDFLKNMSSVKNNIDNIKKEMSKITVCGKAGSNIVVIEMDGEFNVKKVSINKEFFDDLDNGAFEQMVKSALNDAVSKVKEEIKLKTMGVLPFGM

SEQ ID NO: 55 - protein, 80 aa, Borrelia afzelii
MDNDEIFSKVRSIISEQLDKKEDEITIDSRFVEDLNADSLDIYELLYLLEEAFDDKIPENEANEFETVGDVVNFIKKRKG

SEQ ID NO: 56 - protein, 80 aa, Borrelia garinii; identical to SEQ ID NO: 55

SEQ ID NO: 57 - protein, 355 aa, Borrelia afzelii
MKIDKLFVKSIILTFLSMNLIFMILIILGDLFVNLLNFLEKNISLKDIFYIYYLYLPKAFSDGVALSFLFAISNLIGNLSMRNEIIGLFSCGVPLTRILRPIILISVFISVVLFFFDNYLVIDTVAKRDLLIKNSIGNNGSIDKTIIIRDLAREIYNIKSYDIDENAFSNLMIIIKDSKDEFQTRYDINKAEWKDNKWKLYGIREFVKVGRKIKENAYDVLDGTGIIKLEPDYIRTVMLSSKALNFTKLINWISFLKSEHLNYSDALFDLLNRIFFSFRLMLLSFTVGFIALALKKNIFILSLLNSIAFAVVYVISIVIFSFLADLGYLNIYVASSFTTIFFLIINFLVYRIVRK

SEQ ID NO: 58 - protein, 355 aa, Borrelia garinii
MKIDMLFVKSIILTFLSMNLLFMILIILGDLFVNLLSYLEKNIGFKDILYIYYLYLPKAFSDGVALSFLFAISNLIGNLSMRNEIIGLFSCGVSLTRILRPIILISLFISFVLFFFDNYLVIDTVARRDLLIKNSIGNSRSSDKTIIIRDLAREIYNIKSYDINENAFSNLMIIIKDSKDEFQTRYDIDKAEWKDNKWRLYGIREFVKVGRKIKENAYDILDGTGIIKLEPDYIRTVMLSSKALNFTKLINWISFLKSEHLNYSDALFDLLNRVFFSFRLILLSFTVGFIALALKKNIFILSLLNSIAFAVVYVISIVIFNFLADLGYLHIYVASSFTTIFFLIINFFVYRIVRK

SEQ ID NO: 59 - protein, 66 aa, Borrelia afzelii
MTKVPLKKIQDFDGNFYELVVATIIRTEQIIDNISLAEHAISDDKILGQAFNDVLTGKFSYSIEGR

SEQ ID NO: 60 - protein, 66 aa, Borrelia garinii
MTKVPLKKIQDFDGNFYELVVATIIRTEQIIDNISLAEHAVFDDKILGQAFNDVLTGKFSYSIEGR

SEQ ID NO: 61 - protein, 90 aa, Borrelia afzelii
MPISKEVDLEEIIKQNVGKSKFAFRVDNDVISNNTSNVKPGRSKMTIKQISIGLKYEYWVEFYSILDKKGLTASGFIRTLIMEYIETSKK

SEQ ID NO: 62 - protein, 91 aa, Borrelia garinii
MPISKEVDLEEIIKQNISKSKFAFRVDNDVVDNNNSNVKPGRSKMTIKQISIGLKYEYWVEFYSILDKKGLTASGFIRTLIMEYIETSKKS

SEQ ID NO: 63 - protein, 254 aa, Borrelia afzelii
MSELNSYSISLDNFTGGIELFFEYVNINRHVLRTLSLNQIIEEFIVVSSDLNISIRGLIDFFYLANNLFYWKTQILFPLKLECKDKAERKVVERLVGYSKNRENEKLQLNQIKNFKFYDLKMIENFIFGNSGNFVKNINTIANDNKKKNSKRKPNVLKEKVINKNSLYNKKFKVVCNKYELKIFEKKMKIISILNKESRVIFDSLLDFNADNYSFERFSYFLVSLECKMLNIVNLGYLNNKLVLYRGCKINDCT

SEQ ID NO: 64 - protein, 249 aa, Borrelia garinii
MSELSSYSISLDNFTGGIELFFEYVSMNRHVLRTLSLNQIIEEFIVVSSDLNISIRGLIDFFYLANNLFYWKTQILFPLKLEYKDKAERKVVERLIDHSKNRENGKLQLNQIKNFKFYDSKMIEDFIFGNSDNFVKNINAISNDNNKKKNLKKKSSVLKEKVINKKFKVVFNKYELKIFEKKMKIISILNKESSVIFDSLLDFNADNYSFERFSYFLVSLECKMLNIINLGYLDTKLVLYRGCKINDCT

SEQ ID NO: 65 - protein, 579 aa, Borrelia afzelii
MKTIKKDPEFYNSLATLKKYIIKYIEEKVLKYSISSQLYKLKKPEIIELIKISNDYEKEKNVSNSSLEEYYYKKTQNEIIKKWILEIVRNKNFTQISKETNFNTKKLGITYKLKSNNFLKLIEIQNNPYYSQEKKDIYKQIILNFSSNINIDNLEQTIDILVAVRNKNKFKILNILNNNLKNQKENQKVFKSSLINKESRLIKLKRILILIYWPVGCLSKNIFIKILIKHYKYLEEEILALKYNEILNYLRAIKTLSLNEIFYKGSSKNINFNYFFSDFTQYTPKDFQDTLKCYLFIVEKTITKKYLTWFFKDKVSNNWESFLYALEYIEKHKLINIKEKIKEIITAKFQTEDFFTSFDKTNFNPYESSILFKDKYIKNVFIQSIMSYVEKNSQNIETYGWIAFYIYADNKDKKIYCQYIKEFFKKKPFEIQNQIFVYFLSFYPNIENKHFKFISDILLYLDENKITIPKKIIKMQKISPNQLDYQRSYLLIKSLKTRSRILKIILKNIRFNLIEKLEDENDKKLLPALYYLIYCENLVELKNNKKIKSNEKSGLLEFISFFKAKLKQKINFIKRAYEV

SEQ ID NO: 66 - protein, 582 aa, Borrelia garinii
MKTIKKDPEFYDSLAKLKKYIIKYIEAKVLKYSISSQLYKLEKPEIIELIKISNDYEKEKNVLNISLEEYYYKKTKNEIIKKWILEIVKNKNFTQISEEANFITKKLGITYKLKSNIFLKLIEIQNNPYYSQEKKDIYKQIILNFSSNINIDSLEQTIDILVAVRNKNKIKILNILNKNLKNQSENQKVFKSSLINKENRLIKLKRMLILTYWPVGCLSKNIFIKILIKYYKYLEEEILALKYNEILNYLRAIKTLSLNEIFYKGSSKNINFNYFFSDFTQYNPKDFQDTLKRYLYVVGKTITKKYLTWFFKDKISNNWESFIYALEYIQKHKLINIKEKIKEIITAKFQTEDFFISFDKTNFNPYEGSIIFKEKYIKNAFIKSIINYVEKNSQNIETYGWIAFYIYADNEDKKFFCQNMKEFFKTKPFEIQNQIFIYFLSFYPNIENKHFKFISDILLYLDENKITIPKKIIKMQKINPNQLDYQKSYLLIKSLKTRSRILKIILKNIRFNLIEKLEDENEKKLLPAICYLIYCENLVKLKNNRKIKSNEKSSLLEFISFFKTKLKQKINFKKELMKSKGI

SEQ ID NO: 67 - protein, 300 aa, Borrelia afzelii
MKEDRIVFIFGPTAVGKSNILFHFPRNKAEIINVDSIQVYKEFNIASSKPGKSLMKHIKHHLVDFLEPEKEYTLGIFYEQALKIVKEIRQKKKIPIFVGGTAFYFKHLKDGFPSTPLVTSKIRIYVNNLLDLKGKSHLLKELKNVDPIRFNMLNKNDIYRIKRSLEVYYQTGIPISQFQKKQNSEFKNILIVGLKRSFEDLKTRISIRINEMLNSGLLSEIKGLFSKGYNENTPAFKGIGYNEFLLWKSRPWYSLNDIISLINKNSVLYAKRQMTFFAKMPDVLWFHPEDDLDDILNLIF

SEQ ID NO: 68 - protein, 306 aa, Borrelia garinii
MKEDRIVFIFGPTAVGKSNILFHFPKNKAEIINVDSIQVYKEFNIASSKPSKNLMKHIKHHLVDFLDPEKEYTLGIFYEQALRIVKEIRQKKKIPIFVGGTAFYFKHLKDGFPSTPLVTSEIRIYVNNLLYLKGKSYLLKELKNVDPIRFNMLNKNDIYRIKRSLEVYYQTGIPISQFEKKQNSEFKNILIIGLKRSFEDLKTRISIRINEMLNSGLLSEIKGLFSKGYNENTPAFKGIGYNEFLLWKSRPCYSLNDIISLINKNSFLYAKRQMTFFAKISNVLWFHPEDDLVSILNLIFNVDKEI

SEQ ID NO: 69 - protein, 413 aa, Borrelia afzelii
MNNYKVLHTSDWHIGKKIENFSILKEQKNFLSFLLEFLKKEKIDLLLVAGDVYDSKRPGFEEQRLVNNFFYELSFTSCKWCVVISGNHDKKDYLSINKKLLSRFNFFLITEYDSDEQIVFLKDNGILKFIVVCFPHINERFILGQNFDNIFGLEDQSSSKLFLENLENAYREKISSLSNFLENKYKGIPKILMAHSFFSSSKKIDTLGGSYIIPFNVFGNGFSYVALGHIHKFMKLRDNIVYSGSPMQYSFNETHDKYINVLHFNDNKLILQKAFPVPIFNKLIFVKGSLNEVLDFLNNVRKDETFTIYLKIELNEAIDTSAEETIYDLAKLNFINLVSISYSLLLSQNLQDDSSDIGELEVLEMDEKYFFEKKLRRDFENGVIKDIKFKEEELISLFNEVLAKGYLGEYEDK

SEQ ID NO: 70 - protein, 413 aa, Borrelia garinii
MSNYKILHTSDWHIGKKIENFSILKEQKKFLSFLLEFIKKEKIDLLLVAGDVYDSKRPGFEEQKLVNNFFYELSFTPCKWCVVISGNHDKKDYLSINKKLLSRFNFFLITEYDSDEQIVFLKDNENLKFIVVCLPHINERLILGQNFDNIFGLEDPSSSKLFLENLENAYREKISSLSNFLENKYKRIPKILMAHSFFGSSKKIDTLGGSYIIPFNVFGNGFSYVALGHIHKFMKLKDNIVYSGSPMQYSFNETHDKYINVLHFNHNKLILQEAFPVPIFNKLIFVKGSLNEVLDFFNNFKKEESFTIYLKIELNEAVDTSAEETIYDLAKLNFINLVSISYSLPSSQDLQDDSSFIGELEVLEMDEKYFFEKKLRRDFENGVIKDIKFKEEELISLFNEVLAKGYLGEYEDK

SEQ ID NO: 71 - protein, 92 aa, Borrelia afzelii
MMDLEKTNKIKVAEHIVECFGGIKNIKNIKKDITRIKILVDSNSLVKRDDLTKNDNIIGTIKSNELTEIVMNFEIIEDVYNKILYMMNDQKQ

SEQ ID NO: 72 - protein, 92 aa, Borrelia garinii
MADIEKTNKIKVAEHIVECFGGIKNIKNISKDITRIKILVDSNSLVKRDDLTKNDNIIGTIKSNELTEIVMNFEIIEDVYNKILYMMNDQKQ

SEQ ID NO: 73 - protein, 1377 aa, Borrelia burgdorferi
MKEIKDFERIKIKIASPDQIRNWSYGEVKKSETINYRTLRPEKDGLFCERIFGTTKEWECYCGKFKSVRYKGIICDRCNVEVTHFKVRRERMGHIELAAPVAHIWYYKYIPSRIGLLLDITASSLNSILYYEKYVVIEPGDTDLKKMQLLNEDEYIEARERYGMSFNASMGAEAIKTLLENLDLDELSSKLRIQMIDKDDKTDKKLLRRLEIIENFKISGNKPEWMIMEVLPVIPPEIRPMVQLDGGRFATSDLNDLYRRVINRNNRLRKLLLLNAPEIIVRNEKRMLQESVDSLFDNSHKRKVVKGSSSRPLKSLSDALKGKQGRFRQNLLGKRVDYSGRSVIVVGPELKLHQCGLPAKMALELFKPFVIRRLIESEAVFNIKRAKNLIEQEVDEVWQILDLVIKEHPILLNRAPTLHRLGIQAFEPVLVEGKAIKLHPLVCHAYNADFDGDQMAVHVPLTPAAQAESWALMLSTNNLLNPANGHPIVFPSQDIVLGLYYLTMEKKNVVGEGKKFLNFNNVILAINNRSLDYNASIYVKIHGEYKKTTAGRVIFNEALPKGIEFVNKTLSDLELQILISKVYVVHGSSIVIEMLDIIKELGFRYATKFGCTISMSDIIVPDEKRTYVERANKEIAKIQNDYAKGVITGEERYNNVVSVWLKTNEELTNKMMEILKKDRDGFNVIYMMADSGARGSRNQIRQLAGMRGLMAKTSGDIIELPIISNFKEGLSVIEFFISTNGARKGLADTALKTADAGYLTRRLVDIAQDVVVRIEDCGTINGIKVETVKNGEEILESLKEKAVGSYSIERIKNPITGEIVLDANEEISEAKIELLEKIGIEKLVIRSVLTCEAEHGVCQKCYGRDFSKNKPVNIGEAVGIIAAQSIGQPGTQLTMRTFHIGGVAQAGSEDDKISLKNAFILNGIEGFNVRVDNGILFTRKGTLKIINVFYEEKIKNIKEIKVLDSQRVIKGIPLFIDKKGSEILSSYIGYVKLRDDNFFIVSEEQEVSLKAGTKLEIEVGDYVESGKVIGTFDPFAEPIIAEVKGKIKFKDIILGTTLKEEINTETGNVEKRITDNVFESLDPRIFIIDSSGMEVASYVLPGDAYLQVEDGQSINIGDIIAKLSKGSEKTQDITGGLPRVNDLFETRIPKNLTEMAKVSGIVQFKSIQKGKRLINILDEYGVEHKHYIPAGKHLLVRDGDVVKAGDMLCDGRINPHDVLEILGGISLQEFLLAEIQDVYRKQGVSINDKHIGVIIKQMMKKVKIVAVGDTNFVYGQKVDKHTFYEQNRKVIEQGGEPAIASPILIGVTKTSLNIDSFISAASFQETTKVLTDASIAGKIDDLRGLKENVVIGHLIPTGTGMGLYKKIKVSENIDSEV

SEQ ID NO: 74 - protein, 1377 aa, Borrelia afzelii
MKEIKDFEKIKIKIASPDQIRNWSYGEVKKSETINYRTLRPEKDGLFCERIFGTTKEWECYCGKFKSVRYKGIICDRCNVEVTHFKVRRERMGHIELAAPVAHIWYYKYIPSRIGLLLDITASSLNSILYYEKYVVIEPGDTDLKKMQLLNEDEYIEARERYGMSFNASMGAEAIKSLLENLDLDELSSKLRIQMIDKDDKTDKKLLRRLEIIENFKISGNKPEWMIMEVLPVIPPEIRPMVQLDGGRFATSDLNDLYRRVINRNNRLRKLLLLNAPEIIVRNEKRMLQESVDSLFDNSHKRKVVKGSSSRPLKSLSDALKGKQGRFRQNLLGKRVDYSGRSVIVVGPELKLHQCGLPAKMALELFKPFVIRRLIESEAVFNIKRAKNLIEQEVDEVWQILDLVIKEHPILLNRAPTLHRLGIQAFEPVLVEGKAIKLHPLVCHAYNADFDGDQMAVHVPLTPSAQAESWALMLSTNNLLNPANGHPIVFPSQDIVLGLYYLTMEKKNTVGEGKKFLNFNNVILAINNRSLDYNASIYVKIDGKYKKTTAGRVIFNEALPNGIEFVNKTLSDLELQILISKVYVVHGSSTVIEMLDIIKELGFKYATKFGCTISMSDIIVPDEKKAYIDRANKEIAKIQNDYAKGVITGEERYNNVVSVWLKTNEELTNKMMEILKKDRDGFNVIYMMADSGARGSRNQIRQLAGMRGLMAKTSGDIIELPIISNFKEGLSVIEFFISTNGARKGLADTALKTADAGYLTRRLVDIAQDVVVRIEDCGTINGIKVETVKNGEEILESLKEKAVGSYSIERIKNPITGEIVLDANEEISEAKIELLEKIGIEKLVIRSVLTCEAEHGVCQKCYGRDFSKNKPVNIGEAVGIIAAQSIGQPGTQLTMRTFHIGGVAQAGSEDDKISLKNTFILNGIEGFNVRVENGILFTRKGTLKIINVFYEEKIKNIKEIKVSDSQRVIKGIPLFVNNKGVEILSSYIGYVKLRDDKFLIVSEEQEVSLKAGTKLEIEVGEYVESGKVIGTFDPFAEPIIAEVKGKIKFKDIILGTTLKEEINTETGNVEKRITDNVFESLDPRIFIIDSGGVEIASYVLPGDAYLQVEDGQNINIGDIIAKLSKGSEKTQDITGGLPRVNDLFETRIPKNLTEMAKVSGIVQFKSIQKGKRLINILDEYGVEHKHYIPAGKHLLVRDGDIVKAGDMLCDGRINPHDVLEILGGISLQEFLLAEIQDVYRKQGVSINDKHIGVIIKQMMKKVKIVAVGDTNFVYGQKVDKHTFYEQNRKVIEQGGEPAIASPILIGVTKTSLNIDSFISAASFQETTKVLTDASIAGKIDDLRGLKENVVIGHLIPTGTGMGLYKKIKVSENVNSEV

SEQ ID NO: 75 - protein, 455 aa, Borrelia burgdorferi
MALAAFSNSALLFNDGAEKAVISSIFYNSDKIEQIAFHVRADDFYNETHKLIFKAMVSLYEKRENIDPITVFEEVSKHVSKTQLLIKKDLINLPDYLDSLSVYLPTDRTINVYAKIVKEQSVRRSILNVSKELNDYINDSTKSINEIVEESQQKILSIELDYSSKNLYHAKVIAERVHNEIYERSMKKKEANFGIPSGFRKVDSLIGGFRNSDFIIVGARPSIGKTAFALNIASYIALRKEEKKKVGFFSLEMTADALIKRIISSQSCIDSFKVQNSILSGQEIKSLNDIINEISDSELYIEDTPNISLLTLATQARKLKRFYGIDIIFVDYISLISFETKNLPRHEQVASISKSLKELARELEIPIVALSQLTRDTEGREPNLASLRESGALEQDADIVILLHRDKDFKFESSAEIEPIETKVIVAKHRNGPTGRADILFLPHITKFVNKDHQY

SEQ ID NO: 76 - protein, 455 aa, Borrelia afzelii
MALAAFSNSALLFNDGAEKAVISSIFYNSDKLEQIAFHVRADDFYNETHKLIFKAMVSLYEKRENIDPITVFEEVSKHVSKTQLLLKKDLINLPDYLDSLSVYLPTDRTINVYAKIVKEQSVRRSILNVSKELNDYISDSTKTINEIVEESQQKILSIELDYSSKNLYHAKVIAERVHNEIYERSMKKKEANFGIPSGFRKVDSLIGGFRNSDFIIVGARPSIGKTAFALNIASAIALRKEGKKKVGFFSLEMTADALIKRIISSQSCIDSFKVQNSILSGQEIKSLNDVVNEISDAELYIEDTPNISLLTLATQARKLKRFYSIDIIFVDYISLISFETKNLPRHEQVASISKSLKELARELEIPIIALSQLTRDTEGREPNLASLRESGALEQDADIVILLHRDKDFKFESSAEIEPIETKVIVAKHRNGPTGRADILFLPHITKFVNKDHQY

SEQ ID NO: 77 - protein, 464 aa, Borrelia burgdorferi
MDVGIYGLGVMGSNLALNIADNGFNVSVYNRDNEKTEIFVKQNSHKKINGFKDIESFVKSLKTPKKIILMVTSSAIEKVIEQILPFMNKSDIIIDGGNSHYKSTMRLEKELFAKDIYFVGLGISGGERGARFGPALMYGGSKSAYEILEPMLNKIAAKTKNNDICSTYIGENGSGHYVKMIHNGVEYADMQLISEVYFFMKKAFNLDNLKISEVFEKWNEGDLSGYLLEITSKILKYKENNEYLVDKILDIANQKGTGVWTAIDALESGVPVNLIVESVFSRFMSGLKHERIIASDLLKMDTSSFEFELSDWILDLYYALLVSKIVAYAQGFMMLKTASVNYGWDLNLGKISLVWREGCIIRSSFLDKIKLAYDKNPHLINLLFDDYFLDLLKNNHKSLRRIISKASEIGIPLPAFYASLSFLDSYSTNYLPSNLIQAQRDFFGAHSFERLDSKRGEFFHSAWQ

SEQ ID NO: 78 - protein, 464 aa, Borrelia afzelii
MDVGIYGLGVMGGNLALNIADNGFNVSVYNRDSEKTEIFVKQNSHKKINGFKDIESFVKSLKTPRKIILMVTSLAVEKVVEQILPFLNKSDIIIDGGNSHYKSTMRLEKELFAKDIYFVGLGISGGERGARFGPALMYGGSKSAYEILEPILDKIAARTKNNDICSAYIGKNGSGHYVKMIHNGVEYADMQLISEVYFFMKKAFNLDNSKISEVFEKWNEGDLSGYLLEITSKILRYKENNEYLVDKILDIANQKGTGVWTSIDALESSMPVNLIIESVFSRFMSGLKHERIIASDLLKMDTVSFEFELSDWILDLYYALLVSKIVAYAQGFMMLKTASLNYSWDLNLGKISLVWREGCIIRSSFLDKIKLAYDKNPHLINLLFDDYFLDLLKNNHKSLRRIVSKASEIGIPLPAFYASLSFLDSYSTNYLPSNLIQAQRDFFGAHSFERLDSKRGEFFHSTWQ

SEQ ID NO: 79 - protein, 436 aa, Borrelia burgdorferi
MSNFFENYLRQVDDIETVSFVGRVQKIKGLLVESLGPQCAIGDLCLIDQRNGKKVCAEVLGFNGPYVSLMAYEGFSGIEVGNKVYSLNKGLEINLSDELLGRVIDSLGRPIDNKGSFLNNSYKELIFEKINPINRSIFEDQILTGVKVLDGFLPVAKGQRVGIFSGSGVGKSTLLGMIAKNSNADVNVIAFIGERGRELNEFIEHELGEERLKKSVLVVSTSDESPISRYKGAYVATMIAEYFREQGKDVALLFDSITRFANAKREMSLSLGEPPVAKGYPPSVFVEIPILLERSGFNGNGGSVTGFYTVLVEGDDFTEPVADNIKAVLDGHIILDRDLFDRGIYPSINVLSSTSRSIHRIMSLEKQKLIMKARNLLSIYKDYEDLIRTGIYLKGSNKDVDFAISKYPKIINFISQGINETFDFENLEDEIKEILS

SEQ ID NO: 80 - protein, 436 aa, Borrelia afzelii
MSNFFENYLRQVGDIETVAFVGRVQKIKGLLVESLGPQCAVGDLCLIDQRNNKKVCAEVLGFNGPYVSLMAYEGFSGIEVGNKVYSLNKGLEINLSDELLGRVIDSLGRPIDNKGSFLNNKYKELIFEKINPISRSIFEEQILTGVKVLDGFLPVAKGQRVGIFSGAGVGKSTLLGMIAKNSNADVNVIAFIGERGRELNEFIEHELGEERLKKSVLVVSTSDESPISRYKGAYVATMIAEYFRDQGKDVALLFDSITRFANAKREMSLSLGEPPVAKGYPPSVFVEIPILLERSGFNGNGGSVTGFYTVLVEGDDFAEPVADNIKAVLDGHVILDRDLFDRGIYPSINILSSTSRSIHRIMSLEKQKLIMKARNLLSVYKDYEDLIRTGIYLKGSNKDVDFAISKYPKIIDFISQGINETFDFENLEDEIRKILS

SEQ ID NO: 81 - protein, 423 aa, Borrelia burgdorferi
  misc_feature (3)..(4): Xaa can be any naturally occurring amino acid
MVXXKTLEPKFFQSLLDNSPTPYHLVNYIEEKLINYFNAQQLKLNEKWKIKTGSYYIKKEGTSLIAFNIDVKKKYEPFLIAAAHTDSPGLKLKIDATEKVSGVFYNHIEVYGSPIISTWIDRDLSLAGIVYFKKNENIESKLINIENIGIIPNLAIHLNRQINEGFKYNAHDNLTVISSTKKAIKDNILEQLGIECENFLSCDLIFTESQPSKIIGTEGEFLASKNLDNKSGCHAIMNSYVHTSNDKNKIAVFFDNEEVGSLTSRGADSNFLSEVLERIDIALDLTREEHLIKTNKSFNISIDSVHGIHPGYTSKHDPNYQANLGKGVVVKNSANFRYATTSTGFAKLKNLAIKNNIKIQEIIMKANVPSGTTIGPISNARTGIETIDIGTPMWAMHSLRETVSIADHIEAIKLLRAFFEKGI

SEQ ID NO: 82 - protein, 423 aa, Borrelia afzelii
MVHDKTLEPKFFQNLLDNNPTPYHLVNYIEEKLINYFNAQQLKLDEKWKIETGSYYIKKEGTSLIAFNIDVEKKYEPFLIASAHTDSPGLKLKIDATEKKGGMFYNHIEVYGSPIISTWIDRDLSLAGIVYFKKNENINSKLINIENIGIIPNLAIHLNRQINEGFGYNTHDNLTVINSTKKTIKENILEQLGIKYENFLSCDLIFTESQPSKIIGTEGEFLASKNLDNKSGCHAIMNSYVHTSNNKNKIAVFFDNEEVGSLTSRGADSNFLSEVLERIDLALNLTREEHLIKTNKSFNISIDSIHGIHPGYAFKHDPNYQATLSKGVVVKNSANFRYATTSTGFAKLKNLAIENNIKIQEIIMKANVPSGTTIGPISNARTGIETIDIGTPMWAMHSLRETVSIADHIEAIKLLRAFFEKGI

SEQ ID NO: 83 - protein, 442 aa, Borrelia burgdorferi
MMRSLYSGVSGLQNHQTRMDVVGNNIANVNTIGFKKGRVNFQDMISQSISGASRPTDARGGTNPKQVGLGMNVASIDTIHTQGAFQSTQKASDLGVSGNGFFILKEGKNLFYTRAGAFDVDSDRHLVNPANGMRIQGWMARDLEGEKVINTASDIEDLIIPIGDKEGAKSTKNVTFACNLDKRLPLIQEGANPADIARGTWVVNKSLYDSFGNVSVLELRVVKDLNTPNLWNATVLINGEQNSNFTLGFDNEGALASLNGQPGQKGDILQIPITFNVLGANVGEVGEQQTVNLKLGTVGSYTDSITQFADSSSTKAIIQDGYGMGYMENYEIDQNGVIVGIYSNGIRRDLGKIALASFMNPGGLAKSGDTNFVETSNSGQVRIGETGLAGLGDIRSGVLEMANVDLAEQFTDMIVTQRGFQANAKTITTSDQLLQELVRLKN

SEQ ID NO: 84 - protein, 442 aa, Borrelia afzelii
MMRSLYSGVSGLQNHQTRMDVVGNNIANVNTIGFKKGRVNFQDMISQSISGASRPTDVRGGTNPKQVGLGMNVAAIDTIHTQGAFQSTQKASDLGVSGNGFFILKEGGNLFYTRAGAFDVDSDRHLVNPANGMRIQGWMARDLEGEKVINTASDIEDLIIPIGDKEGAKSTKNVTFACNLDKRLPVIQEGASSADIARGTWVVNKSLYDSFGNVSVLELRVVKDPNTPNLWNATVLINGEQNSNFTLGFDNEGALASLNGQPSQKGDVLQIPITFNVLGANVGEVGEQQTVNLKLGTVGSYTDSITQFADASSTKAIIQDGYGMGYMENYEIDQNGIIVGIYSNGIRRDLGKIALASFMNPGGLAKSGDTNFVETSNSGQARIGETGLAGLGDIRSGVLEMANVDLAEQFTDMIVTQRGFQANAKTITTSDQLLQELVRLKN

SEQ ID NO: 85 - protein, 447 aa, Borrelia burgdorferi
MYRIKNENLDFKIDSLGECKQNNPLIDFYASEGSSHFVNEKNKIKFSVYRNEDKGDRYEDVLLEKAGPREKIYFVPRHVKAAITTCGGLCPGFNDVIRSIVRTLWKIYGVRNIYGVKFGYQGLLPESNSPFINLNPDVVDDINKFGGTILGSSRGGIKPVEIVDTLERMNINMIFNIGGDGTQKGSLLIAEEIEKRNLKIAVVGIPKTVDNDFMFVQKSFGFETAVEQAVAAVAGAHFEANSAYNGIGLVKVMGRDSGFIAAHTALSSNDVNFCLIPELDFDIEGPNGFLVHLERRLLEKESLEEIPHAVILIAEGAGQKYFDHFPKKKDDSGNLLYEDIGLYIKDKITEYFKAKNIQFTLKYIDPSYIIRSSPANASDSLYCARLGSNAVHAAMAGKTKMLISLWSTKFVHIPIKMAVIDRNKVNPNGSFWRDVLSSTGQPISMKN

SEQ ID NO: 86 - protein, 447 aa, Borrelia afzelii
MYRIKNENLDFKIDSLGECKQNNPLIDFYASEGSSHFVNEKNKIKFSVYRNEDEGDKYEDVLLEKAGPREKIYFVPRHVKAAITTCGGLCPGFNDVIRSIVRTLWKIYGVRNIYGVKFGYQGLLPESNSPFINLNPDVVDDINKFGGTILGSSRGGIKPVEIVDTLERMNINMIFNIGGDGTQKGSLLIAEEIEKRNLKIAVVGIPKTVDNDFMFVQKSFGFETAVEQAVAAVAGAHFEANSAYNGIGLVKVMGRDSGFIAAHTALSSNDVNFCLIPELDFDIEGPNGFLVHLERRLLEKESLEEIPHAVILIAEGAGQKYFDHFPRKKDDSGNLLYEDIGLYIKDKITEYFKAKNIQFTLKYIDPSYIIRSSPANASDSLYCARLGSNAVHAAMSGKTKMLISLWSTKFVHIPIKMAVIDRNKVNPNGSFWRDVLSSTGQPISMKN

SEQ ID NO: 87 - protein, 264 aa, Borrelia afzelii
MNNFLIKLLLFLLVFSNSYVAFSKNSNVLIITAMDSEVEQINKLMSNKEEIVLKEYGLNKKILKGKLSNRNVITIVCGVGKVNAGVWTSYILSKYNISHVINSGVAGGVVSDKYKDIKVGDVVVSSEVAYHDVDLTKFGHKVGQLMGLPQKFSANKNLVDKATKAVKSKVASSNSYSGLILSGDQFIDPAYINKIIGNFKDVIAVEMEGAAVGHVSHMFNVPFIVIRSISDIVNKEGNELEYSKFLKLAAFNSAKVVQEILRIL

SEQ ID NO: 88 - protein, 264 aa, Borrelia garinii
MNNFLRKFFLFLLVFSNGYVAFSKNINVLIVTAMDSELEEINKLMSNKEEIVLKEYGLNKKIVKGKLSNRNVMAIVCGVGKVNAGVWTSYILSKYNISHVINYGVAGGVISDKYKDIKVGDIVVSSEIAYHDVDLTKFGYKMGQLMGLPQKFIANKNLVNKAKEAVKLKVRGSNAYSGLILTGDQFIDPIYIKKIIRDFKDVIAVEMEGAAIGHVAHMLNVPFVVIRSISDIVNKEENEVEYSKFLKLASFNSAKVVQEILRIL

SEQ ID NO: 89 - protein, 178 aa, Borrelia afzelii
MNKFLIVVLLAFCVFSSFAQADDSKSAFNLGAGEKLLAYETSKKDPIVPFLLNLFLGFGIGSFAQGDILGGFLILGFDAVGIGLIFTGAYLDIKALDKNAPKAAFKWTWGKGMMLAGAVTMAVTRLTEIIIPFTFANSYNRKLKNSLNIAFGGFEPSFDINMGQTSALGFELSFKKSY

SEQ ID NO: 90 - protein, 177 aa, Borrelia garinii
MNKFLIFILVTFCAFSSFAQDDSKSTFNLGVGEKFLVYETNKKDSLVPFLLNLFLGFGIGSFAQGDILGGSLILGFDAVGIGLILTGAYLDIKAFDNNAKKADFKWTWGKGMMLAGVVTMAVTRLTEIVLPFTFANNYNRKLKNSLNIALGGFEPSFDINMGQASALGFGLSFKKSY

SEQ ID NO: 91 - protein, 541 aa, Borrelia afzelii
MSTSSASISIFTILQKVGKAFMLPIALLPAAGILLGIGGAFTNETMIQAYGLEGILGKGTVASSILYLMKYTGEVIFANLPLMFAAAIPIGLAKVEKGTAALAGVVGFLVMHQTINGILYIQGITPESASLKALLELGMPETAATAKSQEYTNVLGIFSLQMSVMGGLVAGFVAVFLHNRFYNIQLPTFLAFFGGTRFVPIITTITMFVVGIFLTFIWPFIQGAMTSFGQIVEQSGLFGTFAYGAIKRSLIPFGLHHIFYLPFWQTAVGGTLEINGELISGAQNIFFKQLGDTNTVHFEVAKGTRFFSGEFVVMIFGLPGAALAMYHTSKPENKKNVASLLLSASFTSMLTGITEPLEFAFLFAAPALYYFIYVPLFGLAYLLTHLLNVGVGLTFSGGFIDMFLFGILQGNSKTNWIAIPILGIFYFIGFYFIFKFVIMKFNLKTIGREDEEMEKDISSEKTNLSETALKVLEGLGGKDNITYLDACASRLRVNLKQIEFIKSDTYFKNLGASGILKKGNSVQIVFGGLSDNIRMEIDKLM

SEQ ID NO: 92 - protein, 542 aa, Borrelia garinii
MSTSSASISIFTILQKVGKAFMLSIALLPAAGILLGIGGAFTNETMIQAYGLEEILGKGSVANSILYLMKYTGEVIFANLPLMFAAAIPIGLAKLEKGTAALAGVVGFLVMHQTINGILYIQGITPESASLKALLELGMPETAATAKSQEYTSVLGIFSLQMSVMGGLIAGFVAVFLHNRFYNIQLPTFLAFFGGTRFVPIITTMTMFVVGIFLTFIWPFIQGAMTSFGNIVEQSGLFGTFVYGAIKRSLIPFGLHHIFYLPFWQTAVGGAMEINGELISGAQNIFFKQLADPNTVHFEVAKGTRFFSGEFVVMIFGLPGAALAMYHTSKPENKKNVASLLISASFTSMLTGITEPLEFAFLFAAPALYYFIYVPLFGLAYLLTHLLNVGVGLTFSGGFIDMFLFGILQGNSKTNWIAIPILGIFYFIGFYFIFKFVIMKFNLKTIGREDEEMEKDEISLEKTNLSETASKVLEGLGGKDNITYIDACASRLRINLKQIEFIKSDAYFKHLGASGMLKKGNSVQIVFGGLSDNIKMEIDKLM

SEQ ID NO: 93 - DNA, 20 bp, Artificial Sequence
  Sense primer for amplifying the gene corresponding to BB0566
aggcctgaaggagagcttgt

SEQ ID NO: 94 - DNA, 24 bp, Artificial Sequence
  Anti-sense primer for amplifying the gene corresponding to protein BB0566
aaacctcatcggatggatactcaa

SEQ ID NO: 95 - DNA, 21 bp, Artificial Sequence
  Sense primer for amplifying the gene corresponding to protein BB0722
gctgattttgccagcgagctt

SEQ ID NO: 96 - DNA, 23 bp, Artificial Sequence
  Anti-sense primer for amplifying the gene corresponding to protein BB0722
tcggtccaaatacttccgtaacc

SEQ ID NO: 97 - DNA, 21 bp, Artificial Sequence
  Sense primer for amplifying the gene corresponding to protein BB0173
tcgcctagtatgggggctgtt

SEQ ID NO: 98 - DNA, 22 bp, Artificial Sequence
  Anti-sense primer for amplifying the gene corresponding to protein BB0173
agcagaaccattgccaagatcc

SEQ ID NO: 99 - DNA, 25 bp, Artificial Sequence
  Sense primer for amplifying the gene corresponding to protein RpoN
aagtgaaaacccccaaaaacaaaaa

SEQ ID NO: 100 - DNA, 25 bp, Artificial Sequence
  Anti-sense primer for amplifying the gene corresponding to protein RpoN
ttgctccaccaacagagctaaaaag

SEQ ID NO: 101 - DNA, 23 bp, Artificial Sequence
  Sense primer for amplifying the gene corresponding to protein Gnd
ggaatgaaggcgatctttcaggg

SEQ ID NO: 102 - DNA, 25 bp, Artificial Sequence
  Anti-sense primer for amplifying the gene corresponding to protein Gnd
gctggcaaaggaatcccaatttcac

(19) United States
(12) Patent Application Publication
(10) Pub. No.: US 2016/0220653 A1
(43) Pub. Date: Aug. 4, 2016

(54) LYME DISEASE VACCINES

(71) Applicants: UNIVERSITÉ DE STRASBOURG, Strasbourg (FR); CENTRE NATIONAL DE LA RECHERCHE SCIENTIFIQUE - CNRS, Paris Cedex 16 (FR)

(72) Inventors: Benoit JAULHAC, Strasbourg (FR); Nathalie BOULANGER, Heiligenstein (FR); Laurence SABATIER, Molsheim (FR); Gilles SCHNELL, Schiltigheim (FR)

(21) Appl. No.: 14/912,325

(22) PCT Filed: Aug. 12, 2014
(86) PCT No.: PCT/FR2014/052085

§ 371 Date: Feb. 16, 2016

(30) Foreign Application Priority Data
Aug. 14, 2013 (FR) 1358017

(51) Int. Cl. A61K 39/02

(52) U.S. Cl. CPC A61K 39/0225; A61K 2039/552

(57) ABSTRACT

The present invention relates to Lyme disease vaccines, in particular to vaccines including one or more isolated polypeptides of Borrelia burgdorferi ss, Borrelia afzelii or Borrelia garinii.

### CROSS REFERENCE TO RELATED APPLICATIONS

This is a U.S. national stage of application No. PCT/FR2014/052085, filed on 12 Aug. 2014. Priority under 35 U.S.C. §119(a) and 35 U.S.C. §365(b) is claimed from French Application No. 13/58017 filed on 14 Aug. 2013, the disclosure of which is also incorporated herein by reference.

### TECHNICAL FIELD

The present invention relates to Lyme disease vaccines, in particular to vaccines comprising one or more isolated polypeptides of Borrelia burgdorferi ss, Borrelia afzelii or Borrelia garinii.

The present invention has applications in the veterinary and medical fields.

In the description below, the references between square brackets ([ ]) refer to the list of references presented at the end of the examples.

### BACKGROUND

Lyme borreliosis, also known as Lyme disease, is a vector-transmitted disease transmitted by a hard tick of the Ixodes genus. It is rife mainly in the Northern hemisphere where it constitutes the most common vector-transmitted disease. Recent data hint at its area of distribution also extending into the Southern hemisphere with human cases in Australia (Mayne et al., 2011 [1]) and ticks infected with Borrelia identified in South America (Barbieri et al., 2013 [2]).

The bacterium responsible for borreliosis is a spirochete belonging to the Borrelia burgdorferi sensu lato group with approximately 20 species identified.

Lyme borreliosis usually develops in the wild fauna in a wide range of vertebrate hosts and manifests itself accidentally in humans first through a skin inflammation, erythema migrans, and then through very varied clinical manifestations: joint, cardiac, neurological and skin manifestations (Radolf et al., 2012 [3]; Stanek et al., 2012 [4]). The skin therefore constitutes an essential interface in the transmission during the tick bite and the development of the disease. The clinical symptoms of borreliosis observed in dogs are very similar to those observed in humans, but more specifically it induces glomerulonephritis in dogs (Little et al., 2010 [5]).

Although antibiotic treatments are effective at an early stage of the infection, many patients develop borreliosis because of an absence or a non-observation of erythema migrans, or because of treatment which is inappropriate or too late. A vaccine approach appears to be more promising for treating or preventing Lyme disease, in particular in animals in which the early skin stage cannot be observed owing to the coat.

There are currently several vaccines on the market for preventing canine borreliosis. In the United States, they are directed only against a single species, Borrelia burgdorferi sensu stricto. Moreover, based on the concept of the “transmission blocking vaccine”, a vaccine was marketed in the United States for humans, LYMErix (trademark), but the marketing of said vaccine was stopped in 2002 following side effects in certain patients (Hanson and Edelman, 2003 [6]). Using a recombinant antigen, OspA (Outer surface protein A), two vaccines are currently used in dogs in the United States (Nobivac (registered trademark) sold by Intervet and Recombitek (registered trademark) sold by Merial) and have shown a certain amount of efficacy, but only against the species B. burgdorferi ss (Lafleur et al., 2009 [7]). Indeed, the Borrelia population transmitted by the ticks is very heterogeneous in Europe, and the vaccines currently on the market are not effective against the other virulent species of Borrelia which are predominant in Europe. A third vaccine is sold using a bacterial lysate of B. burgdorferi ss (Fort Dodge). In Europe, only two companies (Merial and Bioveta) sell a vaccine also based on lysates of Borrelia, B. burgdorferi ss for Merial and B. afzelii and B. garinii for Bioveta. For these various vaccines, three injections are generally required in order to obtain sufficient protection, generally controlled by measuring the antibodies by ELISA (Topfer and Straubinger, 2007 [8]). However, the use of bacterial lysate as a vaccine base is not satisfactory for mass production.

Thus, the existing vaccines use either bacterial lysates of which the mass production is difficult to carry out, or the recombinant protein OspA, which is not very immunogenic and not very highly expressed at the beginning of the infection in humans, or OspC, the proof of concept of which from a vaccine point of view has not been established.

There are therefore real needs to develop novel vaccines which overcome these defects, drawbacks and obstacles of the prior art, in particular vaccines which are strongly immunogenic and effective on various Borrelia populations, and which can be mass produced at low cost.

### SUMMARY

The present invention precisely meets the abovementioned needs of the prior art, by providing vaccine compositions for the prevention of Lyme disease.

For this purpose, the inventors have developed a proteomic approach in order to identify and select polypeptides which are effective for preventing Lyme disease. This approach has been carried out on the basis of three Borrelia species that the inventors have determined as being the most involved in human and animal pathology, in particular in dogs, namely Borrelia burgdorferi ss, Borrelia afzelii and Borrelia garinii.

Thus, a subject of the present invention is in particular a vaccine composition comprising at least one polypeptide of Borrelia burgdorferi ss, Borrelia afzelii or Borrelia garinii, chosen from the sequences described hereinafter.

### DETAILED DESCRIPTION

In particular, the sequences SEQ ID NOs: 1 to 92 are described, presented in table 1 below, in which appear the numbers of sequences of the appended sequence listing, the names of the corresponding polypeptides and the names of the corresponding loci in the genome of Borrelia burgdorferi ss, Borrelia afzelii or Borrelia garinii.

In other words, table 1 describes the isolated polypeptides consisting of a sequence chosen from SEQ ID NOs: 1 to 92 which are likely to be used in the vaccine composition of the invention.

TABLE 1
Sequences of the polypeptides that can be used in a vaccine
composition according to the invention

| | | Name of | | | | Name of |
|---|---|---|---|---|---|---|
| | SEQ | the locus in | SEQ | Name of the | SEQ | the |
| | ID | B. burgdorferi | ID | locus in | ID | locus in |
| Polypeptide name | NO | ss | NO | B. afzelii | NO | B. garinii |
| n/a | 1 | BB0213 | — | — | — | — |
| UDP-N- | 2 | BB0304 | — | — | — | — |
| acetylmuramoylalanyl- | | | | | | |
| D-glutamyl-2,6- | | | | | | |
| diaminopimelate--D- | | | | | | |
| alanyl-D-alanine ligase | | | | | | |
| (murF) | | | | | | |
| fibronectin/fibrinogen- | 3 | BB0347 | — | — | — | — |
| binding protein, | | | | | | |
| putative | | | | | | |
| penicillin-binding | 4 | BB0718 | — | — | — | — |
| protein (pbp-2) | | | | | | |
| n/a | 5 | BB0761 | — | — | — | — |
| n/a | 6 | BB0823 | — | — | — | — |
| outer membrane | 7 | BB0167 | — | — | — | — |
| protein (tnp50) | | | | | | |
| alanine racemase (alr) | 8 | BB0160 | — | — | — | — |
| hemolysin III (yplQ) | 9 | BB0117 | — | — | — | — |
| n/a | 10 | BB0566 | 11 | BAPKO0596 | 12 | BG0576 |
| n/a | 13 | BB0173 | 14 | BAPKO0175 | 15 | BG0172 |
| n/a | 16 | BB0722 | 17 | BAPKO0766 | 18 | BG0744 |
| 50 S ribosomal protein | 19 | BB0229 | — | — | 20 | BG0232 |
| L31 type B | | | | | | |
| flagellar hook-basal | 21 | BB0292 | — | — | 22 | BG0295 |
| body protein FliE | | | | | | |
| Beta glucosidase, | 23 | BB0620 | — | — | 24 | BG0639 |
| putative | | | | | | |
| 30S ribosomal protein | 25 | BB0491 | — | — | 26 | BG0503 |
| S14 type Z | | | | | | |
| n/a | 27 | BB0765 | — | — | 28 | BG0788 |
| n/a | 29 | BB0748 | 30 | BAPKO0794 | — | — |
| Preprotein translocase | 31 | BB0395 | 32 | BAPKO0410 | — | — |
| subunit SecE | | | | | | |
| Holo ACP synthase | 33 | BB0010 | 34 | BAPKO009 | — | — |
| n/a | 35 | BB0029 | 36 | BAPKO028 | — | — |
| n/a | 37 | BB0081 | 38 | BAPKO0081 | — | — |
| n/a | 39 | BB0102 | 40 | BAPKO0103 | — | — |
| RNA polymerase | 41 | BB0450 | 42 | BAPKO0472 | — | — |
| sigma-54 factor | | | | | | |
| Type III pantothenate | 43 | BB0527 | 44 | BAPKO0553 | — | — |
| kinase | | | | | | |
| tRNA (guanine-N(1)-)- | 45 | BB0698 | 46 | BAPKO0742 | — | — |
| methyltransferase | | | | | | |
| n/a | — | — | 47 | BAPKO0189 | 48 | BG0186 |
| n/a | — | — | 49 | BAPKO0265 | 50 | BG0258 |
| flagellar biosynthesis | — | — | 51 | BAPKO0285 | 52 | BG0278 |
| protein FliP | | | | | | |
| Nucleoid associated | — | — | 53 | BAPKO0491 | 54 | BG0475 |
| protein EbfC | | | | | | |
| Acyl carrier porter | — | — | 55 | BAPKO0747 | 56 | BG0726 |
| (ACP) | | | | | | |
| n/a | — | — | 57 | BAPKO0861 | 58 | BG0834 |
| n/a | — | — | 59 | BAPKO0873 | 60 | BG0845 |
| n/a | — | — | 61 | BAPKO4502 | 62 | BGP215 |
| n/a | — | — | 63 | BAPKO0132 | 64 | BG0132 |
| n/a | — | — | 65 | BAPKO0215 | 66 | BG0210 |
| tRNA dimethylallyl- | — | — | 67 | BAPKO0874 | 68 | BG0846 |
| transferase | | | | | | |
| exonuclease SbcD | — | — | 69 | BAPKO0882 | 70 | BG0854 |
| Hypothetical protein | — | — | 71 | BAPKO0366 | 72 | BG0366 |
| RNA polymerase | 73 | BB0388 | 74 | BAPKO0029 | — | — |
| subunit beta | | | | | | |
| replicative DNA | 75 | BB0111 | 76 | BAPKO0112 | — | — |
| helicase | | | | | | |
| 6-phosphogluconate | 77 | BB0561 | 78 | BAPKO0590 | — | — |
| dehydrogenase | | | | | | |
| flagellum-specific ATP | 79 | BB0288 | 80 | BAPKO0298 | — | — |
| synthase FliI | | | | | | |
| putative | 81 | BB0627 | 82 | BAPKO0669 | — | — |
| aminopeptidase 2 | | | | | | |
| flagellar hook protein | 83 | BB0283 | 84 | BAPKO0293 | — | — |
| FlgE | | | | | | |
| phosphofructokinase | 85 | BB0727 | 86 | BAPKO0771 | — | — |
| 5- | — | — | 87 | BAPKO0619 | 88 | BG0601 |
| methylthioadenosine/S- | | | | | | |
| adenosylhomocysteine | | | | | | |
| nucleosidase, putative | | | | | | |
| n/a | — | — | 89 | BAPKO0034 | 90 | BG0034 |
| PTS system, maltose | — | — | 91 | BAPKO0027 | 92 | BGB26 |
| and glucose-specific | | | | | | |
| IIABC component | | | | | | |

In the present invention, unless otherwise indicated, “Borrelia burgdorferi” signifies “Borrelia burgdorferi ss” or “Borrelia burgdorferi sensu stricto”, as opposed to the indication “Borrelia burgdorferi sensu lato” which covers approximately 20 different species.

In addition, unless otherwise indicated, the letters determining the sequences of the polypeptides hereby described correspond to the one-letter abbreviation proposed by Leder (Leder et al. Introduction to molecular medicine, Ed Scientific American, 1994 [9]).

Thus, a subject of the present invention is in particular a vaccine composition comprising at least one polypeptide of Borrelia burgdorferi ss, Borrelia afzelii or Borrelia garinii chosen from the sequences SEQ ID NO: 1 to 92. Preferably, the at least one polypeptide is chosen from the sequences SEQ ID NOs: 10 to 92, preferably 10 to 18, preferably 10 to 15.

Any polypeptide of sequence SEQ ID NOs: 1 to 92, or any combination of at least two of the polypeptides of sequences SEQ ID NOs: 1 to 92, can be used in the vaccine composition according to the invention.

For example, the combination of polypeptides may comprise 2 different polypeptides of sequence SEQ ID NOs: 1 to 92, for example 3, 4, 5, 6, 7, 8, 9 or even more than 9 different polypeptides of sequence SEQ ID NOs: 1 to 92. A combination of the different polypeptides of sequence SEQ ID NOs: 1 to 92 may in fact make it possible to increase the therapeutic and/or prophylactic effect of the vaccine composition according to the invention. For example, the vaccine composition according to the invention may comprise a combination of two polypeptides as presented in table 2 below.

TABLE 2
Combination of polypeptides in the vaccine composition
according to the invention

| Polypeptide | |
|---|---|
| of sequence | Possible combination with the polypeptides of sequence |
| SEQ ID NO: | SEQ ID NO: as follows |
| 1 | At least one from: 2 to 92, |
| | preferably at least one from: 10 to 18, 47 to 72 and 87 to 92 |
| 2 | At least one from: 1 and 3 to 92, |
| | preferably at least one from: 10 to 18, 47 to 72 and 87 to 92 |
| 3 | At least one from: 1, 2 and 4 to 92, |
| | preferably at least one from: 10 to 18, 47 to 72 and 87 to 92 |
| 4 | At least one from: 1 to 3 and 5 to 92, |
| | preferably at least one from: 10 to 18, 47 to 72 and 87 to 92 |
| 5 | At least one from: 1 to 4 and 6 to 92, |
| | preferably at least one from: 10 to 18, 47 to 72 and 87 to 92 |
| 6 | At least one from: 1 to 5 and 7 to 92, |
| | preferably at least one from: 10 to 18, 47 to 72 and 87 to 92 |
| 7 | At least one from: 1 to 6 and 8 to 92, |
| | preferably at least one from: 10 to 18, 47 to 72 and 87 to 92 |
| 8 | At least one from: 1 to 7 and 9 to 92, |
| | preferably at least one from: 10 to 18, 47 to 72 and 87 to 92 |
| 9 | At least one from: 1 to 8 and 10 to 92, |
| | preferably at least one from: 10 to 18, 47 to 72 and 87 to 92 |
| 10 | At least one from: 1 to 9 and 11 to 92, |
| | preferably at least one from: 13 to 18 |
| 11 | At least one from: 1 to 10 and 12 to 92, |
| | preferably at least one from: 13 to 18 |
| 12 | At least one from: 1 to 11 and 13 to 92, |
| | preferably at least one from: 13 to 18 |
| 13 | At least one from: 1 to 12 and 14 to 92, |
| | preferably at least one from: 10 to 12 and 16 to 18 |
| 14 | At least one from: 1 to 13 and 15 to 92, |
| | preferably at least one from: 10 to 12 and 16 to 18 |
| 15 | At least one from: 1 to 14 and 16 to 92, |
| | preferably at least one from: 10 to 12 and 16 to 18 |
| 16 | At least one from: 1 to 15 and 17 to 92, |
| | preferably at least one from: 10 to 15 |
| 17 | At least one from: 1 to 16 and 18 to 92, |
| | preferably at least one from: 10 to 15 |
| 18 | At least one from: 1 to 17 and 19 to 92, |
| | preferably at least one from: 10 to 15 |
| 19 | At least one from: 1 to 18 and 20 to 92, |
| | preferably at least one from: 10 to 18 and 29 to 92 |
| 20 | At least one from: 1 to 19 and 21 to 92, |
| | preferably at least one from: 10 to 18 and 29 to 92 |
| 21 | At least one from: 1 to 20 and 22 to 92, |
| | preferably at least one from: 10 to 18 and 29 to 92 |
| 22 | At least one from: 1 to 21 and 23 to 92, |
| | preferably at least one from: 10 to 18 and 29 to 92 |
| 23 | At least one from: 1 to 22 and 24 to 92, |
| | preferably at least one from: 10 to 18 and 29 to 92 |
| 24 | At least one from: 1 to 23 and 25 to 92, |
| | preferably at least one from: 10 to 18 and 29 to 92 |
| 25 | At least one from: 1 to 24 and 26 to 92, |
| | preferably at least one from: 10 to 18 and 29 to 92 |
| 26 | At least one from: 1 to 25 and 27 to 92, |
| | preferably at least one from: 10 to 18 and 29 to 92 |
| 27 | At least one from: 1 to 26 and 28 to 92, |
| | preferably at least one from: 10 to 18 and 29 to 92 |
| 28 | At least one from: 1 to 27 and 29 to 92, |
| | preferably at least one from: 10 to 18 and 29 to 92 |
| 29 | At least one from: 1 to 28 and 30 to 92, |
| | preferably at least one from: 10 to 28, 47 to 72 and 87 to 92 |
| 30 | At least one from: 1 to 29 and 31 to 92, |
| | preferably at least one from: 10 to 28, 47 to 72 and 87 to 92 |
| 31 | At least one from: 1 to 30 and 32 to 92, |
| | preferably at least one from: 10 to 28, 47 to 72 and 87 to 92 |
| 32 | At least one from: 1 to 31 and 33 to 92, |
| | preferably at least one from: 10 to 28, 47 to 72 and 87 to 92 |
| 33 | At least one from: 1 to 32 and 34 to 92, |
| | preferably at least one from: 10 to 28, 47 to 72 and 87 to 92 |
| 34 | At least one from: 1 to 33 and 35 to 92, |
| | preferably at least one from: 10 to 28, 47 to 72 and 87 to 92 |
| 35 | At least one from: 1 to 34 and 36 to 92, |
| | preferably at least one from: 10 to 28, 47 to 72 and 87 to 92 |
| 36 | At least one from: 1 to 35 and 37 to 92, |
| | preferably at least one from: 10 to 28, 47 to 72 and 87 to 92 |
| 37 | At least one from: 1 to 36 and 38 to 92, |
| | preferably at least one from: 10 to 28, 47 to 72 and 87 to 92 |
| 38 | At least one from: 1 to 37 and 39 to 92, |
| | preferably at least one from: 10 to 28, 47 to 72 and 87 to 92 |
| 39 | At least one from: 1 to 38 and 40 to 92, |
| | preferably at least one from: 10 to 28, 47 to 72 and 87 to 92 |
| 40 | At least one from: 1 to 39 and 41 to 92, |
| | preferably at least one from: 10 to 28, 47 to 72 and 87 to 92 |
| 41 | At least one from: 1 to 40 and 42 to 92, |
| | preferably at least one from: 10 to 28, 47 to 72 and 87 to 92 |
| 42 | At least one from: 1 to 41 and 43 to 92, |
| | preferably at least one from: 10 to 28, 47 to 72 and 87 to 92 |
| 43 | At least one from: 1 to 42 and 44 to 92, |
| | preferably at least one from: 10 to 28, 47 to 72 and 87 to 92 |
| 44 | At least one from: 1 to 43 and 45 to 92, |
| | preferably at least one from: 10 to 28, 47 to 72 and 87 to 92 |
| 45 | At least one from: 1 to 44 and 46 to 92, |
| | preferably at least one from: 10 to 28, 47 to 72 and 87 to 92 |
| 46 | At least one from: 1 to 45 and 47 to 92, |
| | preferably at least one from: 10 to 28, 47 to 72 and 87 to 92 |
| 47 | At least one from: 1 to 46 and 48 to 92, |
| | preferably at least one from: 1 to 46 and 73 to 86 |
| 48 | At least one from: 1 to 47 and 49 to 92, |
| | preferably at least one from: 1 to 46 and 73 to 86 |
| 49 | At least one from: 1 to 48 and 50 to 92, |
| | preferably at least one from: 1 to 46 and 73 to 86 |
| 50 | At least one from: 1 to 49 and 51 to 92, |
| | preferably at least one from: 1 to 46 and 73 to 86 |
| 51 | At least one from: 1 to 50 and 52 to 92, |
| | preferably at least one from: 1 to 46 and 73 to 86 |
| 52 | At least one from: 1 to 51 and 53 to 92, |
| | preferably at least one from: 1 to 46 and 73 to 86 |
| 53 | At least one from: 1 to 52 and 54 to 92, |
| | preferably at least one from: 1 to 46 and 73 to 86 |
| 54 | At least one from: 1 to 53 and 55 to 92, |
| | preferably at least one from: 1 to 46 and 73 to 86 |
| 55 | At least one from: 1 to 54 and 56 to 92, |
| | preferably at least one from: 1 to 46 and 73 to 86 |
| 56 | At least one from: 1 to 55 and 57 to 92, |
| | preferably at least one from: 1 to 46 and 73 to 86 |
| 57 | At least one from: 1 to 56 and 58 to 92, |
| | preferably at least one from: 1 to 46 and 73 to 86 |
| 58 | At least one from: 1 to 57 and 59 to 92, |
| | preferably at least one from: 1 to 46 and 73 to 86 |
| 59 | At least one from: 1 to 58 and 60 to 92, |
| | preferably at least one from: 1 to 46 and 73 to 86 |
| 60 | At least one from: 1 to 59 and 61 to 92, |
| | preferably at least one from: 1 to 46 and 73 to 86 |
| 61 | At least one from: 1 to 60 and 62 to 92, |
| | preferably at least one from: 1 to 46 and 73 to 86 |
| 62 | At least one from: 1 to 61 and 63 to 92, |
| | preferably at least one from: 1 to 46 and 73 to 86 |
| 63 | At least one from: 1 to 62 and 64 to 92, |
| | preferably at least one from: 1 to 46 and 73 to 86 |
| 64 | At least one from: 1 to 63 and 65 to 92, |
| | preferably at least one from: 1 to 46 and 73 to 86 |
| 65 | At least one from: 1 to 64 and 66 to 92, |
| | preferably at least one from: 1 to 46 and 73 to 86 |
| 66 | At least one from: 1 to 65 and 67 to 92, |
| | preferably at least one from: 1 to 46 and 73 to 86 |
| 67 | At least one from: 1 to 66 and 68 to 92, |
| | preferably at least one from: 1 to 46 and 73 to 86 |
| 68 | At least one from: 1 to 67 and 69 to 92, |
| | preferably at least one from: 1 to 46 and 73 to 86 |
| 69 | At least one from: 1 to 68 and 70 to 92, |
| | preferably at least one from: 1 to 46 and 73 to 86 |
| 70 | At least one from: 1 to 69 and 71 to 92, |
| | preferably at least one from: 1 to 46 and 73 to 86 |
| 71 | At least one from: 1 to 70 and 72 to 92, |
| | preferably at least one from: 1 to 46 and 73 to 86 |
| 72 | At least one from: 1 to 71 and 73 to 92, |
| | preferably at least one from: 1 to 46 and 73 to 86 |
| 73 | At least one from: 1 to 72 and 74 to 92, |
| | preferably at least one from: 10 to 28, 47 to 72 and 87 to 92 |
| 74 | At least one from: 1 to 73 and 75 to 92, |
| | preferably at least one from: 10 to 28, 47 to 72 and 87 to 92 |
| 75 | At least one from: 1 to 74 and 76 to 92, |
| | preferably at least one from: 10 to 28, 47 to 72 and 87 to 92 |
| 76 | At least one from: 1 to 75 and 77 to 92, |
| | preferably at least one from: 10 to 28, 47 to 72 and 87 to 92 |
| 77 | At least one from: 1 to 76 and 78 to 92, |
| | preferably at least one from: 10 to 28, 47 to 72 and 87 to 92 |
| 78 | At least one from: 1 to 77 and 79 to 92, |
| | preferably at least one from: 10 to 28, 47 to 72 and 87 to 92 |
| 79 | At least one from: 1 to 78 and 80 to 92, |
| | preferably at least one from: 10 to 28, 47 to 72 and 87 to 92 |
| 80 | At least one from: 1 to 79 and 81 to 92, |
| | preferably at least one from: 10 to 28, 47 to 72 and 87 to 92 |
| 81 | At least one from: 1 to 80 and 82 to 92, |
| | preferably at least one from: 10 to 28, 47 to 72 and 87 to 92 |
| 82 | At least one from: 1 to 81 and 83 to 92, |
| | preferably at least one from: 10 to 28, 47 to 72 and 87 to 92 |
| 83 | At least one from: 1 to 82 and 84 to 92, |
| | preferably at least one from: 10 to 28, 47 to 72 and 87 to 92 |
| 84 | At least one from: 1 to 83 and 85 to 92, |
| | preferably at least one from: 10 to 28, 47 to 72 and 87 to 92 |
| 85 | At least one from: 1 to 84 and 86 to 92, |
| | preferably at least one from: 10 to 28, 47 to 72 and 87 to 92 |
| 86 | At least one from: 1 to 85 and 87 to 92, |
| | preferably at least one from: 10 to 28, 47 to 72 and 87 to 92 |
| 87 | At least one from: 1 to 86 and 88 to 92, |
| | preferably at least one from: 1 to 46 and 73 to 86 |
| 88 | At least one from: 1 to 87 and 89 to 92, |
| | preferably at least one from: 1 to 46 and 73 to 86 |
| 89 | At least one from: 1 to 88 and 90 to 92, |
| | preferably at least one from: 1 to 46 and 73 to 86 |
| 90 | At least one from: 1 to 89, 91 and 92, |
| | preferably at least one from: 1 to 46 and 73 to 86 |
| 91 | At least one from: 1 to 90 and 92, |
| | preferably at least one from: 1 to 46 and 73 to 86 |
| 92 | At least one from: 1 to 91, |
| | preferably at least one from: 1 to 46 and 73 to 86 |

The vaccine composition according to the invention may comprise, in addition to any of the combinations of two polypeptides presented in table 2 above, at least one third polypeptide different than those of the combination, or even a fourth different polypeptide, etc. Preferably, the vaccine composition comprises a combination of 2, 3, 4, 5, 6 or 7 different polypeptides.

The polypeptides that can be used in the vaccine composition according to the invention are not limited to the polypeptides consisting of the sequence SEQ ID NOs: 1 to 92. Those skilled in the art clearly understand that sequences exhibiting a homology or an identity with these sequences can also be used, in an equivalent manner, in the vaccine composition according to the invention, provided that they have the same effect as the polypeptides of sequence SEQ ID NOs: 1 to 92, namely an immunogenic effect, of use in the prevention of Lyme disease. Those skilled in the art are able to identify homologous sequences from the sequences of the polypeptides that can be used in the vaccine composition according to the invention. For example, a sequence used can have greater than 80% homology or identity with a sequence described in table 1, for example greater than 85%, or than 90%, or than 95%, or than 99% identity or homology with a sequence described in table 1. Various methods, well known to those skilled in the art, can be used to determine the homology between several sequences. This may, for example, be the BLAST (Basic Local Alignment Search Tool) method described in the document Altschul, S. F. et al., J. Mol. Biol. 1990 [27].

In table 1 above, the polypeptide sequences which appear on the same line represent the same protein, the name of which is indicated in the left-hand column. “n/a” signifies that the name of the protein in question has not been identified or is not yet known.

Although the polypeptides which appear on the same line represent the same protein, their amino acid sequences are not strictly identical. This may be due to the possible mutations which have occurred distinctly in the various species of the Borrelia genus. By way of examples, the sequences SEQ ID NOs: 10, 11 and 12, isolated from the species Borrelia burgdorferi ss, Borrelia afzelii and Borrelia garinii, respectively, represent the same protein.

Polypeptides that can be used in the vaccine composition according to the invention may be specific to a given Borrelia species, or common to two or three Borrelia species chosen from Borrelia burgdorferi ss, Borrelia afzelii and Borrelia garinii. For example:
the polypeptides of sequence SEQ ID NOs: 1 to 9 are proteins specific to the virulent clone of Borrelia burgdorferi ss;
the polypeptides of sequence SEQ ID NOs: 10 to 18 are proteins common to the virulent clones of Borrelia burgdorferi ss, Borrelia afzelii and Borrelia garinii;
the polypeptides of sequence SEQ ID NOs: 19 to 28 are proteins common to the virulent clones of Borrelia burgdorferi ss and Borrelia garinii,
the polypeptides of sequence SEQ ID NOs: 29 to 46 are proteins common to the virulent clones of Borrelia burgdorferi ss and Borrelia afzelii;
the polypeptides of sequence SEQ ID NOs: 47 to 72 are proteins common to the two virulent clones of Borrelia afzelii and Borrelia
the polypeptides of sequence SEQ ID NOs: 73 to 86 are proteins common to Borrelia burgdorferi ss and Borrelia afzelii; and
the polypeptides of sequence SEQ ID NOs: 87 to 92 are proteins common to Borrelia afzelii and Borrelia garinii.

The polypeptides of sequence SEQ ID NOs: 1 to 9, 13 to 18, 29 to 32, 37, 38, 51, 52, 57, 58, 71, 72 and 87 to 92 are membrane proteins of Borrelia.

Advantageously, when the composition comprises a combination of polypeptides of sequence SEQ ID NOs: 1 to 92, the polypeptides are polypeptides of different sequences. Likewise advantageously, when the composition comprises a combination of polypeptides of sequence SEQ ID NOs: 1 to 92, the polypeptides represent different proteins.

Advantageously, when the composition comprises a combination of polypeptides of sequence SEQ ID NOs: 1 to 92, the polypeptides may be proteins the combination of which makes it possible to obtain an immunization simultaneously against two of the species or the three species Borrelia burgdorferi ss, Borrelia afzelii and Borrelia garinii. In other words, advantageously, the composition according to the invention comprises a protein common to the abovementioned three species or a mixture of several proteins, for example, 2, 3 or 4 proteins, or even more, covering these three species. This embodiment makes it possible to provide vaccine compositions which are universal with respect to the Borrelia populations.

According to one particular embodiment, the vaccine composition according to the invention may comprise at least one polypeptide of Borrelia burgdorferi ss, Borrelia afzelii or Borrelia garinii chosen from SEQ ID NOs: 2, 7 to 15, 19, 20, 25, 26, 29, 30, 33 to 36, 41 to 50, 53, 54, 57 to 72 and 85 to 92. Preferably, the at least one polypeptide is chosen from SEQ ID NOs: 10 to 15.

In this embodiment, the vaccine composition may also comprise at least one other polypeptide of Borrelia burgdorferi ss, Borrelia afzelii or Borrelia garinii chosen from SEQ ID NOs: 1, 3 to 6, 16 to 18, 21 to 24, 27, 28, 31, 32, 37 to 40, 51, 52, 55, 56 and 73 to 84.

In addition, in this embodiment, the vaccine composition may also comprise at least one other polypeptide of Borrelia burgdorferi, Borrelia afzelii or Borrelia garinii chosen from the groups (c1), (c2) and (c3),

said group (c1) comprising SEQ ID NOs: 19 to 28,

said group (c2) comprising SEQ ID NOs: 29 to 46 and 73 to 86,

said group (c3) comprising SEQ ID NOs: 47 to 72 and 87 to 92,

on the condition that, if said at least one polypeptide chosen from SEQ ID NOs: 2, 7 to 15, 19, 20, 25, 26, 29, 30, 33 to 36, 41 to 50, 53, 54, 57 to 72 and 85 to 92:
is included in one of the groups (c1), (c2) or (c3), said at least one other polypeptide is included in a different group (c1), (c2) or (c3), or
is SEQ ID NO: 2, 7, 8 or 9, said at least one other polypeptide is included in the group (c3), or
is SEQ ID NO: 10, 11, 12, 13, 14 or 15, said at least one other polypeptide is included in any of the groups (c1), (c2) and (c3).

According to another embodiment, the vaccine composition comprising at least one polypeptide of Borrelia burgdorferi ss, chosen from the sequences SEQ ID NOs: 10, 2, 8, 9, 13, 19, 25, 29, 33, 35, 43, 45 and 85. Preferably, the at least one polypeptide is chosen from SEQ ID NO: 10.

In this other embodiment, the vaccine composition may also comprise at least one other polypeptide of Borrelia burgdorferi ss, Borrelia afzelii or Borrelia garinii chosen from SEQ ID NOs: 1, 3 to 7, 11, 12, 14 to 18, 20 to 24, 26 to 28, 30 to 32, 34, 36 to 42, 44, 46 to 84 and 86 to 92.

In addition, in this other embodiment, the vaccine composition may also comprise at least one other polypeptide of Borrelia burgdorferi, Borrelia afzelii or Borrelia garinii chosen from the groups (c1), (c2) and (c3),

said group (c1) comprising SEQ ID NOs: 19 to 28,

said group (c2) comprising SEQ ID NOs: 29 to 46 and 73 to 86,

said group (c3) comprising SEQ ID NOs: 47 to 72 and 87 to 92,

on the condition that, if said at least one polypeptide chosen from SEQ ID NOs: 10, 2, 8, 9, 13, 19, 25, 29, 33, 35, 43, 45 and 85:
is SEQ ID NO: 19 or 25, said at least one other polypeptide is included in the group (c2) or (c3), or
is SEQ ID NO: 29, 33, 35, 43, 45 or 85, said at least one other polypeptide is included in the group (c1) or (c3), or
is SEQ ID NO: 2, 8 or 9, said at least one other polypeptide is included in the group (c3), or
is SEQ ID NO: 10 or 13, said at least one other polypeptide is included in any of the groups (c1), (c2) and (c3).

Advantageously, the composition of the invention may comprise a pharmaceutically acceptable carrier.

In the present text, the term “pharmaceutically acceptable carrier” is intended to mean any substance which makes it possible to dilute or transport at least one polypeptide of the vaccine composition according to the invention. Preferably, the pharmaceutically acceptable carrier does not affect the efficacy of the polypeptide. The pharmaceutically acceptable carrier may, for example, be an aqueous solution or an emulsion.

When the pharmaceutically acceptable carrier is an aqueous solution, said solution may be, for example, any one of the solutions presented in the document Heitz et al., 2009 (Twenty years of cell-penetrating peptides: from molecular mechanisms to therapeutics. Heitz F et al., Br J Pharmacol. 2009 May; 157(2):195-206 [10]) or in the document Wehrlé P. (Wehrlé P., Pharmacie galénique, Formulation et technologie pharmaceutiques [Galenical pharmacy, Pharmaceutical formulation and technology], 2007 [11]).

When the pharmaceutically acceptable carrier is an emulsion, said emulsion may be a water-in-oil, oil-in-water or water-in-oil-in-water emulsion (Wehrlé P. [11]).

The vaccine composition according to the invention may comprise an adjuvant. The term “adjuvant” is intended to mean any substance capable of facilitating and amplifying the immune response to the at least one polypeptide of the vaccine composition according to the invention. It may for example be any adjuvant known to those skilled in the art for the administration of polypeptides.

For example, the adjuvant may be an adjuvant which induces a humoral response and/or a cellular response. By way of nonlimiting examples, the adjuvant may be chosen from alumina hydroxide, saponin extracts, immune stimulating complexes (also called “ISCOMs”), inulin, Toll receptor (TLR) agonists, Cytosine Phosphate Guanine (CPG) complexes, chitosan or else mycolic acids. It may also be saponin (Roatt et al., 2012 [14]) or alumina hydroxide (Livey et al., 2011 [15]; Wressnigg et al. 2013 [16]).

The vaccine composition according to the present invention may be used alone or in combination with any known treatment for preventing Lyme disease and/or one or more pathological condition(s) distinct from Lyme disease. By way of example, the pathological condition(s) distinct from Lyme disease may be chosen from the group comprising leptospirosis, rabies, distemper, parvoviral infection and Bordetella infections.

According to the invention, the term “used in combination” is intended to mean a use of the vaccine composition according to the invention jointly or simultaneously, concomitantly, or successively, with any known treatment for preventing Lyme disease. The mode of administration may be identical or different according to the molecules coadministered.

The term “jointly or simultaneously” is intended to mean the use of the composition according to the invention with any known treatment for preventing Lyme disease in a single composition containing them.

The term “concomitantly” is intended to mean the separate use of the vaccine composition according to the invention and of any known treatment for preventing Lyme disease, via identical or different routes of administration during the same administration period.

The term “successively” is intended to mean the separate use of the vaccine composition according to the invention and of any known treatment for preventing Lyme disease, via identical or different routes of administration during different administration periods.

The term “administration period” is intended to mean the period of time during which a treatment is administered. It may, for example, be several days, for example two days, three days, four days, etc., for example one or more weeks, for example one week, two weeks, three weeks, etc., for example one or more months, for example one month, two months, three months, etc., for example one or more years, for example one year, two years, three years, etc.

The present invention also relates to a vaccine composition according to the invention, for use as a medicament.

The present invention also relates to a vaccine composition according to the invention, for use in the prevention of Lyme disease.

The vaccine composition according to the invention may therefore be used for the production of a medicament, in particular a medicament intended for preventing Lyme disease.

The vaccine composition for use as a medicament or for use in the prevention of Lyme disease may be intended for any mammal capable of contracting or having contracted Lyme disease. In particular, it may be intended for human beings or for dogs, for horses, for cattle or for other ruminants. Preferably, the vaccine composition according to the invention is intended for the prevention of Lyme disease in dogs.

The vaccine composition according to the invention, used as a medicament, may be in any appropriate administration form. It may be one of the forms known by those skilled in the art for administering an active molecule which is a polypeptide (Peppas N A, Carr D A, Chemical engineering Science, 64, 4553-4565 (2009) [12]; Morishita M, Peppas N A, Drug Discovery Today, 11, 905-910 (2006) [13]).

The vaccine composition according to the present invention may, for example, be intended for administration by injection.

Thus, the vaccine composition according to the invention may be packaged in any form known to those skilled in the art for the purpose of being administered by injection. It may, for example, be a bottle or a vial.

For example, the injection may be an intramuscular, intradermal or subcutaneous injection. According to one embodiment, the injection is carried out intradermally. According to another advantageous embodiment, the injection is carried out intramuscularly or subcutaneously.

The vaccine composition of the present invention may be administered as a medicament, preferably in sufficient amount to prevent Lyme disease, in particular for preventing Lyme disease in dogs. For example, the polypeptides of the vaccine composition according to the invention may be inoculated at doses of between 1 and 500 μg, preferably between 10 and 100 μg (Wressnigg et al., 2013 [16]).

The synthesis of the polypeptides that can be used in the vaccine composition according to the present invention may be carried out by any process known to those skilled in the art. It may for example be a synthesis by genetic engineering.

When the synthesis of the polypeptides of the vaccine composition according to the invention is carried out by genetic engineering, it is for example possible to construct a large polypeptide comprising the polypeptide of the vaccine composition of the present invention and to digest it with restriction enzymes in order to collect said polypeptide of the vaccine composition according to the invention. The protocol described in F. Cordier-Ochsenbein et al. J. Mol. Biol. 279, 1177-1185 [17] can, for example, be used.

The vaccine composition according to the invention may be produced according to any method well known to those skilled in the art. It may for example be simple mixing of the various constituents of the vaccine composition. The document by Ramamoorthi and Smooker (2009) [18] describes a process for producing a vaccine composition that can be used in the context of the present invention.

Thus, the present invention provides effective solutions for the prevention of Lyme disease.

Other advantages may become further apparent to those skilled in the art on reading the examples below given by way of nonlimiting illustration.

### BRIEF DESCRIPTION OF THE FIGURES

FIG. 1 represents the PCR quantification of B. burgdorferi, native strain 297 and of its virulent clone 297c4, in mouse skin, relative to the time after inoculation of the strain. “N Fla/10⁴ GAPDH” signifies the number of flagellin per 10⁴ of glyceraldehyde 3-phosphate dehydrogenase.

FIG. 2 represents an expression profile, by RT-PCR, of a protein common to the three species of Borrelia: B. burgdorferi ss, B. afzelii and B. garinii, namely BB0566 (SEQ ID NO: 10) in mouse skin during early transmission of the bacterium.

### EXAMPLES

### Example 1

### Strategy for Identifying Borrelia Vaccine Candidates for Lyme Disease

The inventors developed a proteomic approach in order to identify and select effective polypeptides for the prevention of Lyme disease.

They selected, by cloning in solid medium (De Martino et al., 2006 [19]), strains of Borrelia burgdorferi ss, Borrelia afzelii and Borrelia garinii that were virulent and nonvirulent on mice. A Gel-LC-MS/MS strategy was used to compare the proteins in each Borrelia culture.

### 1.1 Materials and Methods

### 1.1.1 Mice, Bacterial Strains and Culture Conditions

C3H/HeN mice three-to-four weeks old were used (Charles River Laboratories, L'Arbresle, France).

Three strains of Borrelia burgdorferi sensu lato were analyzed:

Borrelia burgdorferi ss, strain 297 (A. Steere, USA), isolated from the cerebrospinal fluid (CSF) of a patient,

B. garinii, strain PBi (B. Wilske, Germany) isolated from the CSF of a patient,

B. afzelii, strain 163/98 (E. Ruzic-Sabljic, Slovenia) isolated from the CSF of a patient.

For each species, the bacteria were cloned on BSK-S. The bacterial clones were then cultured on BSK-H medium (Sigma) and tested on a mouse model according to the protocol described in De Martino et al. [19]. The bacterial clones were selected for their virulence in mice. All the strains were cultured in complete BSK-H medium (Sigma) at 33° C. and used at low passage (<7). The Borrelia were counted and the viability was verified by dark-background microscopy.

1.1.2 Identification of Proteins, after Digestion with Trypsin and nanoLC-MS/MS

For each strain and its clone, the proteins were extracted with a Laemmli buffer [20]. After sonication and centrifugation, the pellet was removed and the protein concentration of the supernatent was determined. The proteins (75 μg) were subjected to a prefractionation step on an SDS-PAGE (12% acrylamide) one-dimensional electrophoresis gel. The resulting lanes were stained with Coomassie blue [21]. Gel bands of 2 mm were systematically manually excised. The digestion of the proteins contained in the gel was carried out as described in Villiers et al. [22], and the (tryptic) peptides obtained were extracted by adding 35 μl of 60% (v/v) of acetonitrile (ACN) and 0.1% (v/v) of HCO₂H. The nanoLC-MS/MS analysis was carried out using a nanoLC-Chip/MS system (Agilent Technologies, Palo Alto, Calif.) coupled to an amaZon ion trap (Bruker, Bremen, Germany). The chromatographic system was composed of a precolumn (40 nl, 5 μm) and of a column (150 mm×75 μm, 5 μm) comprising the same Zorbax 300SB-C18 stationary phase. The solvent system was composed of 2% of ACN, 0.1% HCO₂H in water (solvent A) and of 2% water, 0.1% HCO₂H in ACN (solvent B). 1 μl of peptide extract ( 1/20 of the total volume) was loaded in duplicate onto the precolumn (in the enrichment column) at a charge flow rate of (a flow speed fixed at) 3.75 μl/min with solvent A (at 100% of solvent A). The elution was carried out at a flow rate of 300 nl/min by application of a linear gradient of 8-40% of solvent B for 30 minutes, followed by a step of 4 min at 70% of solvent B, before reconditioning of the column with 8% of solvent B. The acquisition parameters of the MS and MS/MS spectra are the following: source temperature regulated at 145° C. and gas flow rate at 4 l/min. The voltage applied to the (nanoelectrospray) sprayer needle was regulated at −1900 V. The acquisition of the MS spectra was carried out in positive (ion) mode over a range of masses of 250 to 1500 m/z at a scanning speed of 8100 m/z per s. The maximum number of ions (control of ionic charge) and the maximum accumulation time were respectively fixed at 200 000 and 200 ms, with an average over two scans. The acquisition of the MS/MS spectra was carried out by sequentially selecting the eight most intense (abundant) precursor ions, with a preference for doubly charged ions. The threshold of selection of an ion for the fragmentation (absolute threshold) was fixed at 100 000. The fragmentation was carried out using argon as collision gas. The ions selected were excluded for 0.6 min. The MS/MS spectra were produced over a mass range of from 100 to 2000 m/z. The maximum number of accumulatable ions in MS/MS (control of ionic charge) was fixed at 400 000, with an average over five scans. The complete system was controlled by the Hystar 3.2 software (Bruker).

### 1.1.3 Data Analysis

The raw data from the MS and MS/MS spectra (mass data collected during the nanoLC-MS/MS) were processed, converted into “.mgf” files with the DataAnalysis 4.0 software (Bruker) and interpreted using the MASCOT 2.4.3 (Matrix Science, London, United Kingdom) [23] and OMSSA 2.1.7 (Open Mass Spectrometry Search Algorithm, Maryland, USA) [24] algorithms. The searches were carried out without any molecular-weight or isoelectric-point restriction in protein data banks composed respectively of sequences (of proteins) of Borrelia burgdorferi ss B31, Borrelia garinii PBi and Borrelia afzelii PKo downloaded from the nonredundant data bank of the National Centre for Biotechnology Information (NCBInr, Aug. 16, 2012). The known contaminating proteins such as human keratin and trypsin were added to each data bank and linked with reverse copies of all the sequences (B. burgdorferi ss B31: 1758 entries; B. garinii PBi: 1720 entries; B. afzelii PKo: 2157 entries). The B. burgdorferi ss B31 and B. afzelii PKo data banks were used since the B. burgdorferi ss 297 and B. afzelii 163 strains respectively have not yet been sequenced. Trypsin was chosen as enzyme. The tolerance with respect to the mass of the precursors and of the fragments was fixed at 0.5 Da. A maximum of two mixed cleavages was accepted and a few post-translational modifications were taken into account: carbamidomethylation (C), N-terminal acetylation, oxidation (M). The results of the MASCOT and OMSSA algorithms were independently loaded into the Scaffold software (Proteome Software, Portland, Oreg.). The level of false positives was fixed at 1% with a minimum of one peptide per protein.

The number of spectra attributed to each protein within each duplicate was used in order to demonstrate proteins overexpressed in the virulent clones. The beta-binomial test [25] was carried out in R in order to determine the overexpression of the proteins (p<0.05) in each virulent clone compared with the wild-type clone. The test was carried out independently for each of the search engines since the spectral identifications are algorithm-dependent.

### 1.2 Results

### 1.2.1 Reproducibility of the Identification of Bacterial Proteins According to the Infection and the Search Algorithm Used

Each sample was analyzed in duplicate. The identification of the proteins present in each repetition was carried out using a combination of the two search engines: Mascot and OMSSA. Thus, the reproducibility of the identifications was evaluated for the two search algorithms. The number of proteins common to the injection duplicates (overlapping) is high (>90% in most cases) and a few proteins were specifically identified in a replicate. This proportion is higher in Mascot compared with OMSSA. These specific identifications could be explained either by the small amount of these proteins, or by the variability due to the data-dependent acquisition (DDA) mode. In order to compare the identifications obtained using the search engines, the identifications of the duplicates were fused. For all the samples, there is a high number (>85%) of (overlapping) proteins identified both by Mascot and by OMSSA. A few proteins are observed specifically by a single algorithm. The combination of the search engines leads to more than 5% of additional identifications.

### 1.2.2 Proteins (Polypeptides) Involved in the Bacterial Transmission

For each species, the identifications obtained by Mascot or

OMSSA were fused and the protein profiles of the wild-type clones and of the virulent clones were compared by the inventors. More than 800 proteins were identified in each case and a significant intersection (overlap) between the wild-type and virulent clones was observed: around 90% for B. burgdorferi ss and B. garinii and 80% for B. afzelii. A higher proportion of proteins detected in the virulent strain compared with the wild-type strain was thus noted for each species (up to 110 for B. burgdorferi ss).

Moreover, proteins present both in the virulent clone and in the wild-type strain of Borrelia, but overexpressed in the virulent clone, were detected. Consequently, a strategy based on the number of spectra attributed to each protein (from spectral counting) coupled to an appropriate statistical test (beta-binomial) [25] was used to demonstrate the proteins overexpressed in the virulent clone compared with the wild-type strain (p<0.05). Statistical tests were carried out independently for Mascot and OMSSA since the number of spectra assigned is generally variable between the two search engines [26]. A protein with a p value less than 0.05 both for Mascot and for OMSSA was considered to be overexpressed in order to limit the number of false positives. The number of proteins overexpressed depends on the species under consideration. 31 overexpressed proteins were detected for B. burgdorferi ss, 43 for B. garinii and 72 for B. afzelii.

The homologous proteins in the three species were determined using the blastp program [27] with an E-value threshold fixed at 10⁻³°. Three proteins are thus common to the three Borrelia species analyzed and 27 proteins are common to at least two of the three species (see table 1 above).

Forty Borrelia proteins were thus retained (represented by the 92 sequences of polypeptides of sequence SEQ ID NOs: 1 to 92 in table 1 above) among more than about a thousand proteins identified for the species belonging to this genus.
Three proteins are common to all the virulent clones and were not detected in wild-type strains: the first, BB0173 (SEQ ID NO: 13) has a von Willebrand type A (VWA) factor domain which is known to be involved in cell adhesion. The second, BB0566 (SEQ ID NO: 10), has a “Sulfate Transporter and Anti-sigma factor antagonist” (STAS) domain. The sigma factors are key elements in the activation of RNA polymerase (RNAP) transcription involved in the regulation of Borrelia. The third protein, BB0722 (SEQ ID NO: 16), has not yet been described in the literature, but appears to be a bacterial membrane-associated protein.
Other proteins are linked to the RNAP catalytic nucleus: the β unit (rpoC) and the sigma-54 factor (RpoN). For example, BAPKO0873 (SEQ ID NO: 59) contains an ω domain of an RNA polymerase subunit (RpoZ). BB0765 (SEQ ID NO: 27) contains a DNA polymerase domain III (DNAX). These proteins are linked to the RpoN-RpoS pathway which plays an important role in microbial pathogenicity and survival (Radolf et al., 2012 [3]). In B. burgdorferi, RpoN directly activates the transcription of RpoS which, in turn, controls the expression of the virulence-associated membrane lipoproteins (OspA, OspC, decorin-binding proteins). A nucleotide-associated protein EBfC appears to be an overall regulator of gene expression in Borrelia. An increase in EBfC levels influences the expression of B. burgdorferi genes by about 4.5%, including genes associated with infection. Other proteins involved in DNA replication, recombination and repair (DNA helicase and SBCD exonuclease), or in tRNA processing, have also been identified.
Four proteins identified relate to periplasmic flagellae: FliE, FliP, FlgE and flagellum-specific ATP synthase (Flip. Mobility is crucial for the infectious cycle of B. burgdorferi and the periplasmic flagellae are essential for providing the bacteria with sufficient mobility. It has been shown that the inactivation of genes encoding flagellar proteins results in non-mobile bacteria. Another study has shown that the loss of flagellae decreases B. garinii infection.
BB0527 (SEQ ID NO: 43) homologous to Baf (Bvg accessory factor) was also identified. This protein has an inhibitory effect on the activity of alkaline phosphatase and therefore directly influences the expression of the P66 outer membrane protein. Among the proteins overexpressed, 5-methylthioadenosine/S-adenosylhomocysteine (SEQ ID NO: 87) was also identified. This protein is an integral part of the methylation cycle. A recent study has shown that the inhibition of this enzyme can attenuate bacterial virulence.
Several proteins identified are involved in carbohydrate metabolism, such as the proteins linked to the phosphotransferase system (PTS), or in the biosynthesis of lipids and metabolism, such as the acyl carrier protein (ACP). Other proteins are hypothetical and do not have a defined function to date.

### Example 2

### Study of Skin Inflammation in Mice, after Inoculations of Various Human Pathotypes of Borrelia burgdorferi Sensu Stricto

The skin constitutes an essential organ in the development of Lyme borreliosis since Borrelia is inoculated therein and multiplies therein before disseminating in the organism and reaching the target organs: joints, nervous system and remote skin.

Various human clinical isolates of B. burgdorferi ss (pathotypes), with various virulence factors of RST type (16S-23S rRNA intergenic spacer type), were selected. The inflammatory responses in the skin in a murine model were compared according to the protocol described hereinafter in order to determine whether the immunity of the skin played a role in the organotropism of the bacterial strains.

The mouse in fact constitutes a model of choice for understanding the pathogenicity mechanisms of B. burgdorferi sl [28].

### 2.1 Materials and Methods

### 2.1.1 Mice and Bacterial Strains

C3H/HeN mice three-to-four weeks old were used (Charles River Laboratories, L'Arbresle, France). The Borrelia burgdorferi sensu stricto strains were isolated from patients suffering from various clinical manifestations: the PBre strain (RST1) from an erythema migrans (EM) (single lesion—Germany), the MR726 strain (RST3) from a multiple erythema migrans (United States), the 1808/03 strain (RST1) from cerebrospinal fluid (Slovenia) and the 297 strain (RST2) also from cerebrospinal fluid (United States). The Borrelia clone c297/4 was selected by culturing on solid BSK medium [19]. All the strains were cultured in complete BSK-H medium (Sigma) at 33° C. and used at low passage (<7). The Borrelia were counted and the viability was verified by dark-background microscopy.

### 2.1.2 Monitoring of the Infection of the Mouse

The mice were infected with 10³ spirochetes in 0.1 ml of BSK medium intradermally in the dorsolumbar region. The control mice were injected with an equal volume of sterile BSK medium and kept under the same conditions as the infected animals. Evaluation of arthritis was carried out every week by measuring the thickness of the two tibiotarsal joints with a metric caliper. Measurements carried out jointly gave an indication of the seriousness of the arthritis. The serology was carried out as described in Kern et al. [29].

At various times after the beginning of the experiment (0 h, 5 h, 24 h, 3 d, 5 d, 7 d, 15 d and 30 d after infection), the mice were killed with an overdose of isoflurane gas. Approximately 1 cm of skin was collected from the site of inoculation and stored in Trizol (registered trademark) (Invitrogen). The ear, the base of the heart, the bladder and the tibiotarsal joints of each mouse were aseptically collected and divided into two parts, for the PCR and the culture of Borrelia. The organs of the noninfected mice were collected under the same conditions as the positive mice.

### 2.1.3 Detection of B. Burgdorferi in the Mouse Organs

For the detection of the spirochetes by culture, the organs removed were placed in 6 ml of BSK-H medium containing 30 μg of rifampicin (BioRad). The tubes were kept at 33° C., and the presence of spirochetes was examined every week by dark-background microscopy.

For the PCR, the DNA was extracted from the organs of each mouse on a MagNA Pure system (Roche Diagnostics, France), using a MagNA Pure LC large-volume isolation kit after external lysis. The heart, the bladder, the ear and the skin were placed in 500 μl of lysis buffer containing proteinase K. Other samples were treated with external lysis using collagenase A, then proteinase K. All the DNA samples were finally eluted in 100 μl of elution buffer. Ten μL of Borrelia DNA were used as a positive control for the detection. The qualitative amplification was carried out as described in Woods et al. [30], by targeting the flagellin gene.

### 2.1.4 Quantification of the Spirochete Load and of the Inflammatory Genes of the Mouse Skin

On the site of inoculation, the B. burgdorferi-specific flagellin gene was quantified on a LightCycler system (Roche Diagnostics, France). The primers used to amplify the fla gene were those described in Kern et al. [29].

To measure the inflammation at the site of inoculation, the total RNA was extracted from 10 mg of mouse skin using the Trizol reagent according to the indications of the manufacturer (Invitrogen). The samples were treated with DNAse (Ambion, USA) and then a first cDNA strand was synthesized from 1 μg of total RNA using the SuperScript II reverse transcriptase (Invitrogen Life Technologies). The gapdh was quantified as an internal standard. The relative expression levels were calculated using an infected animal as calibrator. The amplification and the detection were carried out with an ABI 7000 system with the thermal profile hereinafter: 95° C. for 10 minutes, 50 cycles of 95° C. for 15 s, at 60° C. for 1 min. The primers used for all the genes studied are described in Kern et al. [29].

2.1.5 Comparison of the Protein Profile of the B. Burgdorferi Strain 297, of the Wild-Type Strain and of the Virulent Clone c297/4

The cultures of B. burgdorferi 297, of the wild-type strain and of the virulent clone c297/4 were suspended in Laemmli buffer [20]. The protocol presented in examples 1.1.2 and 1.1.3 above was then applied.

2.1.6 Dynamics of the Genes of B. Burgdorferi 297, of the Wild-Type Strain and of the Virulent Clone c297/4, in the Mouse Skin at the Site of Inoculation

At various times (0 h, 5 h, 24 h, 3 d, 5 d, 7 d, 15 d and 30 d after infection), skin samples were taken from each mouse at the site of inoculation. The total RNA was purified using the Trizol reagent according to the instructions of the manufacturer. The concentration and the purity of the extracted RNAs were determined by measuring the optical density at A260 and A280. The samples were then treated with gDNAse (Qiagen) in order to remove the contamination with DNA. The total RNAs extracted were subjected to Quantiscript Reverse Transcription (Qiagen) so as to produce the cDNA. The cDNA was used to quantify the ospC and bbk32 genes. For B. burgdorferi C297/4, the selected genes corresponding to cell envelope proteins were retained for the RT-PCR. The relative expression levels were calculated using the ΔΔCt method with flagellin as internal standard. The amplification and the detection were carried out with an ABI 7500 system with the thermal profile hereinafter: 95° C. for 10 minutes, 50 cycles of 95° C. for 15 s, at 50° C. for 30 s and 60° C. for 1 min. Each amplification condition was compared on day 3 for the relative quantification. The correlation factors were calculated by comparing the cDNA amplification of each point of the time course of the native strain with the cDNA amplification of each point of the hypervirulent clone. Next, the curve obtained for the clone was standardized with these factors so as to obtain a second curve, representative of the wild-type strain and quantitatively comparable to the clone.

### 2.1.7 Statistical Analysis

Each experiment was carried out at least three times. For each of the RT-PCRs, at least two extractions were carried out for each mouse in each experiment, with two to three mice for each point.

### 2.2 Results

### 2.2.1 Transmission and Diffusion of the Various Pathotypes of B. Burgdorferi ss in Mice

All the strains studied showed a similar diffusion tendency. Borrelia was detected on day 3 by PCR in the skin, on the site of inoculation, for all the strains. They diffused rapidly into the joint, where they were first detected on day 5 or 7, then to the heart and the bladder on day 5, 7 or 15, and the slowest dissemination occurred in the ear (skin remote from the site of inoculation). The PBre pathotype, isolated from erythema migrans (EM) diffused more slowly to the heart and the bladder, compared with the others. By ELISA, all the mice became positive for the Borrelia antigens 15 days after the bacterial inoculation.

### 2.2.2 Quantification of the Borrelia Pathotypes and Measurement of the Inflammation at the Site of Inoculation

The bacterial load of the skin was measured. All the strains multiplied intensively on day 7, but without any significant difference observed between the strains tested.

The inflammatory profile in the skin of the mice was compared for these various strains of B. burgdorferi ss. The antimicrobial peptides (AMPs), which are markers of the innate immunity of epithelia, were measured. The PBre strain (EM) induced a significant amount of cathelicidin with a peak on day 3. The MR726 strain (MEM) strongly induced the defensin mBD-3. The wild-type 297 strain (CSF) exhibited an mBD-3 peak at 24 h while the 1808/03 strain (CSF) induced a negligible amount of all of the three AMPs tested. The induction of supplementary pro-inflammatory molecules was then measured: TNF-α, IL-6, IL-22 and the chemokine MCP-1. For each of them, a TNF-α and/or MCP-1 induction peak was observed on day 7. The MR726 strain isolated from an MEM lesion induced the strongest inflammatory profile in the mouse skin with an MCP-1 peak (150 times) on day 7.

### 2.2.3 Specific Analysis of the Inflammation Induced by B. Burgdorferi ss 297 Wild-Type Strain and its Hypervirulent Clone

The Borrelia infection could be initiated with a heterogeneous population of Borrelia in the vertebrate host. A B. burgdorferi ss 297 clone C297/4 was selected in the laboratory for its rapid diffusion and its neurological manifestations in mice [31]. The virulent clone C297/4 caused an inflammation of the skin with a greater induction of the defensins, MBD-14, and of cathelicidin compared with the wild-type strain. A very strong induction of MCP-1 and of IL-6, approximately 100 times more induction, was observed for the clone C297/4 compared with the wild-type strain.

The results of the wild-type 297 strain and of the hypervirulent clone were also compared in the C3H/HeN mice. The hypervirulent clone diffused more rapidly to the joint, whereas the diffusion to the other organs was similar to that of the wild-type strain. The quantification of the bacterial load in the tissues confirmed the intense multiplication occurring in the skin on day 7 regardless of the strain used, but no significant difference was observed between the hypervirulent clone and the wild-type 297 strain.

2.2.4 Proteomic Characterization of the Wild-Type 297 B. burgdorferi ss Strains and of the Hypervirulent Clone

The protocol presented in examples 1.2.1 and 1.2.2 above was applied.

A total of 887 proteins were identified with 848 c297/4 proteins and 777 proteins in the wild-type strain. An overlap of 738 proteins, which represents 83% of the total number, was observed. 110 proteins are specific for the hypervirulent clone.

2.2.5 Comparative Expression of the Proteins Specific for the Wild-Type and Hypervirulent Clones of B. burgdorferi 297 in the Mouse Skin by RT-PCR

The kinetics of expression of OspC and BBK32, two important proteins in the transmission of Borrelia, were determined for the wild-type and c297/4 strains. The two strains exhibit a first OspC expression peak on day 5, while a BBK32 expression peak was observed on day 7. Borrelia surface proteins were then selected among the 110 proteins specific for the hypervirulent clone, and their expression was monitored during the skin inflammation in the C3H/HeN mouse. Three genes are strongly expressed in the two strains, bb0304, bb0213 and bb0347 with an expression peak on day 5 for the hypervirulent clone and on day 7 for the wild-type strain.

### Example 3

### Selection of the Vaccine Candidates and Determination of the Immunogenic Effect

The various proteins were tested in a C3H/HeN murine model in order to see their expression in the skin, during the transmission of the bacterium. This is because the skin interface appears to play a key role in the selection of certain bacterial populations (Brisson et al., 2011 [32]). The skin of the intradermally infected mice is sampled at 3, 5, 7 and 15 days. After having designed specific primers for each of the proteins, the RT-PCR technique is used to monitor the expression of these proteins in the skin. Those which are the most expressed in the skin are then retained. They are then cloned (Steere et al., 1998 [33]; Ramamoorthi and Smooker, 2009 [18]; Livey et al., 2011 [15]) and expressed in E. coli. They are inoculated intradermally into the mouse and the antibody titer is measured by ELISA. Indeed, the antibody response appears to be essential for measuring a protective effect during Lyme borreliosis (Embers and Narasimhan, 2013 [34]). Their protective effect is tested by means of a challenge with Borrelia inoculated with a syringe, or better still with ticks infected with Borrelia. The vaccinated and challenged mice are then dissected and their organs cultured or tested by PCR in order to measure the absence of Borrelia (Kern et al., 2011 [29]).

Five proteins were retained for in vivo tests in mice, namely the three “hypothetical proteins” only detected in the virulent clones and common to the three Borrelia species (SEQ ID NOs: 10 (BB0566), 13 (BB0173) and 16 (BB0722) of Borrelia burgdorferi ss and the respective corresponding sequences SEQ ID NOs: 11 (BAPKO0596), 14 (BAPKO0175) and 17 (BAPKO0766) of B. afzelii and 12 (BG0576), 15 (BG0172) and 18 (BG0744) of B. garinii), the RpoN protein (SEQ ID NO: 41 (BB0450) of Borrelia burgdorferi ss and the corresponding sequence SEQ ID NO: 42 (BAPKO0472) of B. afzelii) and the Gnd protein (SEQ ID NO: 77 (BB0561) of Borrelia burgdorferi ss and the corresponding sequence SEQ ID NO: 42 (BAPKO0590) of B. afzelii).

In parallel, in the skins of infected mice 7 days after intradermal inoculation, all the Borrelia proteins expressed in the skins of infected mice were analyzed by a non-targeted proteomic approach. This is because 7 days corresponds to a peak of intense multiplication of the bacteria after intradermal inoculation and therefore probably plays a key role in the initiation of the bacterial infection (Kern et al., 2011 [29]). The strategy consisted in extracting the proteins contained in the infected skins, in prefractionating them by gel electrophoresis and then in identifying them by liquid chromatography coupled to tandem mass spectrometry (Ge-LC-MS/MS strategy).

### 3.1 Materials and Methods

### 3.1.1 Inoculation of Bacteria and Sampling of Infected Skins

C3H/HeN mice three-to-four weeks old were purchased from Charles River Laboratories (L'Arbresle, France).

The inventors were particularly interested in the B. burgdorferi ss 297 strain, isolated from cerebrospinal fluid in the United States (Sterre et al., 1893 [35]).

Various clones of B. burgdorferi ss were obtained by culturing on a solid BSK medium ([19]). The 297c4 clone was selected for its rate of dissemination in mice and its location in the brain in particular.

All the Borrelia strains were cultured in BSK-H medium (Sigma) at 33° C. and used at low passage (<7) for the mouse infection. The spirochetes were counted and the viability was verified using a dark-background microscope. The mice were infected with 10³ spirochetes in 0.1 ml of BSK by intradermal injection in the dorsal thoracic region.

At various points after the inoculation (3 d, 5 d, 7 d, 15 d), the mice were killed with isoflurane. An area of 1 cm of mouse skin was collected at the site of inoculation and stored in Trizol (Invitrogen) for the RT-PCR analyses. For the quantification of the Borrelia in the skin, the sample is stored dry at −80° C.

3.1.2 PCR Quantification of the Skins Infected with Borrelia

At the site of inoculation, the detection of the presence of B. burgdorferi ss was carried out by PCR by targeting the flagellin gene on a LightCycler system (Roche Diagnostics, France). The primers used to amplify the fla gene are those previously described ([29]).

### 3.1.3 RT-PCR on the Mouse Skins for Gene Detection

At the various points of the time course, the skin samples were taken from each mouse at the site of inoculation. The total RNA was purified using the Trizol reagent according to the instructions of the manufacturer. The concentration and the purity of the RNA extracted were determined by measuring the A260 and A280. The samples were then treated with gDNAse wipeout (Qiagen). The total RNA extracted was synthesized to give cDNA using Quantiscript reverse transcription (Qiagen). The cDNA was used to quantify the bbk32 genes (positive control). For B. burgdorferi ss 297 and 297c4, the genes corresponding to the three common proteins and RpoN and Gnd were tested by RT-PCR using the primers described in table 3 below.

TABLE 3
Primers used for the RT-PCR

| | SEQ | |
|---|---|---|
| | ID | |
| Proteins | NO: | Sequences |
| BB0566 | 93 | F-AGG CCT GAA GGA GAG CTT GT |
| | 94 | R-AAA CCT CAT CGG ATG GAT ACT CAA |
| BB0722 | 95 | F-GCT GAT TTT GCC AGC GAG CTT A |
| | 96 | R-TCG GTC CAA ATA CTT CCG TAA CC |
| BB0173 | 97 | F-TCG CCT AGT ATG GGG GCT GTT |
| | 98 | R-AGC AGA ACC ATT GCC AAG ATC C |
| RpoN | 99 | F-AAG TGA AAA CCC CCA AAA ACA AAA A |
| | 100 | R-TTG CTC CAC CAA CAG AGC TAA AAA G |
| Gnd | 101 | F-GGA ATG AAG GCG ATC TTT CAG GG |
| | 102 | R-GCT GGC AAA GGA ATC CCA ATT TCAC |

The relative expression levels were calculated using the ΔΔCt method with flagellin as internal standard. The amplification and the detection were carried out with an ABI 7500 system with the following thermal profile: 95° C. for 10 min, 50 cycles of 95° C. for 15 s, and 60° C. for 1 min. Each amplification condition was compared on day 3 for the relative quantification.

### 3.1.4 Proteomic Analysis of the Infected Skins

The biopsies were selected according to the quantification by PCR. Fragments of approximately 4 mg were cut up and the proteins were extracted in 200 μl of Laemmli buffer and then assayed. The proteins (50 μg) were prefractionated on an SDS-PAGE electrophoresis gel and then the migration lanes were excised and treated as described in Example 1. The tryptic peptides were analyzed by nanoLC-MS/MS using the nanoLC-Chip/MS system coupled to the amaZon ion trap, as described in Example 1. The MS and MS/MS spectra were acquired with the same parameters and the searches were performed in the same way, except for the data banks. In the present case, the searches were performed in data banks composed of B. burgdorferi ss B31 and mouse sequences, downloaded from the NCBInr and UniProtKB-SwissProt data bank, respectively (B. burgdorferi B31: Aug. 16, 2012; mouse: Apr. 19, 2013).

### 3.2 Results

### 3.2.1 Borrelia Multiplication Peak on Day 7:

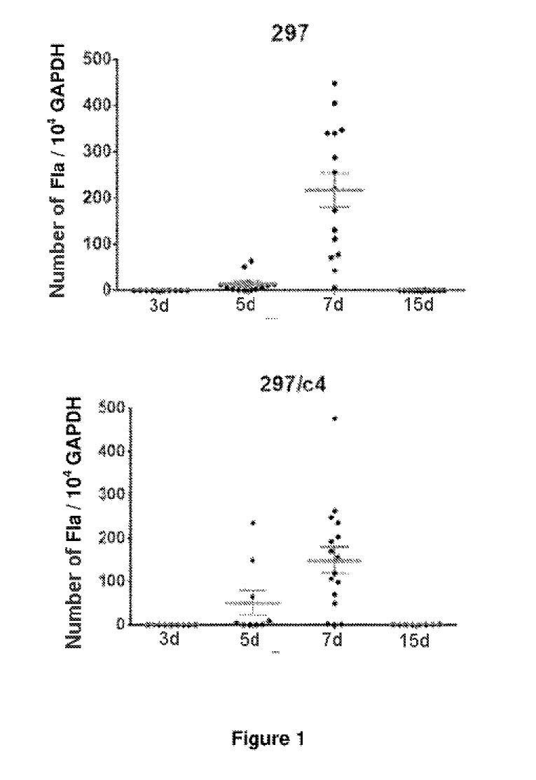

The quantification of the Borrelia in the skin at various points after intradermal inoculation reveals an intense multiplication of the bacteria on day 7, this being regardless of the Borrelia strain tested (FIG. 1).

### 3.2.2 Kinetics of Expression of Certain Genes in the Skin

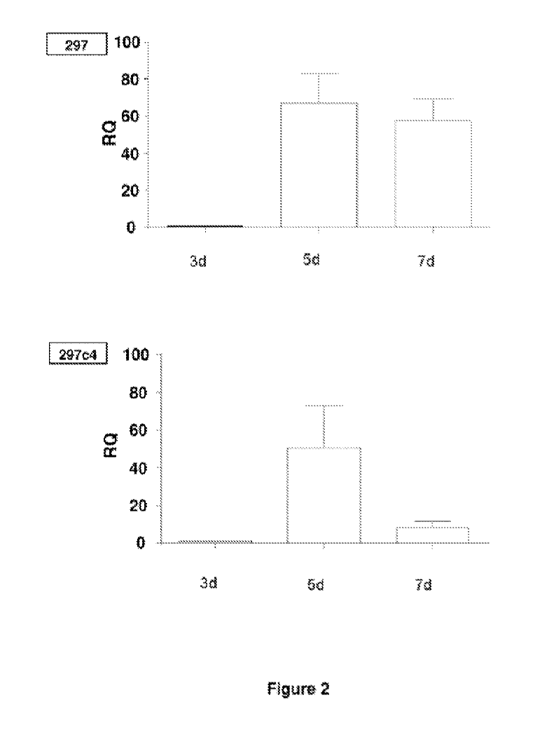

The expression profile, by RT-PCR, of the BB0566 protein (SEQ ID NO: 10), which is a protein common to the three species of Borrelia: B. burgdorferi ss, B. afzelii and B. garinii, in the mouse skin during the early transmission of the bacterium is represented in FIG. 2.

These results show that the protein of sequence SEQ ID NO: 10 is strongly overexpressed in the mouse skin from the fifth day after inoculation for the two strains 297 and 297c4.

3.2.3 Proteomic Analysis of the Skins of Mice Infected on Day Seven after Intradermal Inoculation

On average, 1350 mouse proteins were identified in the skin biopsies from infected mice. Among the Borrelia proteins detected, the RpoN and Gnd proteins were identified, thereby confirming the expression of these proteins in the skin seven days after inoculation and their potential role during the early transmission of the bacterium.

### Example 4

### Vaccine Trial

The dose of recombinant proteins to be administered is determined according to a prior dose-effect study well known to those skilled in the art, generally between 1 and 500 μg. According to the vaccination protocol in dogs, ideally, two administrations will be carried out, 2 to 4 weeks apart, followed by an annual booster. The adjuvant is chosen according to its ability to stimulate the humoral response and/or the cellular response. Those skilled in the art know how to determine which adjuvant to choose in order to efficiently stimulate the humoral response and/or the cellular response. The vaccine is administered intradermally, subcutaneously or intramuscularly, preferably intramuscularly or subcutaneously.

### LIST OF REFERENCES

[1] Mayne P J. Emerging incidence of Lyme borreliosis, babesiosis, bartonellosis, and granulocytic ehrlichiosis in Australia. Int J Gen Med. 2011; 4:845-52.
[2] Barbieri A M et al. Borrelia burgdorferi sensu lato infecting ticks of the ixodes ricinus complex in Uruguay: first report for the Southern Hemisphere. Vector Borne Zoonotic Dis. 2013 March; 13(3):147-53.
[3] Radolf J D et al. Of ticks, mice and men: understanding the dual-host lifestyle of Lyme disease spirochaetes. Nat Rev Microbiol. 2012 Jan. 9; 10(2):87-99.
[4] Stanek G et al. Lyme borreliosis. Lancet. 2012 Feb. 4; 379(9814):461-73. doi: 10.1016/S0140-6736(11)60103-7.
[5] Little S E et al. Lyme borreliosis in dogs and humans in the USA. Trends Parasitol. 2010; 26:213-8.
[6] Hanson M S, Edelman R. Progress and controversy surrounding vaccines against Lyme disease. Expert Rev Vaccines. 2003; 2:683-703.
[7] LaFleur R L et al. Bacterin that induces anti-OspA and anti-OspC borreliacidal antibodies provides a high level of protection against canine Lyme disease. Clin Vaccine Immunol. 2009; 16:253-9.
[8] Töpfer K H, Straubinger R K. Characterization of the humoral immune response in dogs after vaccination against the Lyme borreliosis agent A study with five commercial vaccines using two different vaccination schedules. Vaccine. 2007; 25:314-26.
[9] Leder et al. “Introduction to molecular medicine”, Ed Scientific American, 1994.
[10] Heitz F et al. “Twenty years of cell-penetrating peptides: from molecular mechanisms to therapeutics”. Br J Pharmacol. 2009 May; 157(2):195-206.
[11] Wehrlé P., Pharmacie galénique, Formulation et technologie pharmaceutiques [Galenical pharmacy, Pharmaceutical formulation and technology], 2007.
[12] Peppas N A and Carr D A, Impact of Absorption and Transport on Intelligent Therapeutics and Nano-scale Delivery of Protein Therapeutic Agents. Chemical Engineering Science, 64, 4553-4565 (2009).
[13] Morishita M and Peppas N A, Is the oral route possible for peptide and protein drug delivery? Drug Discovery Today, 11, 905-910 (2006).
[14] Roatt B M et al. Performance of LBSap vaccine after intradermal challenge with L. infantum and saliva of Lu. longipalpis: immunogenicity and parasitological evaluation. PLoS One. 2012; 7(11):e49780. doi: 10.1371/journal.pone.0049780. Epub 2012 Nov. 26.
[15] Livey I et al. A new approach to a Lyme disease vaccine. Clin Infect Dis. 2011 February; 52 Suppl 3:s266-70.
[16] Wressnigg N et al. Safety and immunogenicity of a novel multivalent OspA vaccine against Lyme borreliosis in healthy adults: a double-blind, randomised, dose-escalation phase ½ trial. Lancet Infect Dis. 2013 August; 13(8):680-9.
[17] Cordier-Ochsenbein et al. (1998). “Exploring the folding pathways of annexin I, a multidomain protein. II. Hierarchy in domain folding propensities may govern the folding process.” J Mol Biol 279(5): 1177-85.
[18] Ramamoorthi J, Smooker P. M (2009) So you need a protein-aguide to the production of recombinant proteins. The Open Veterinary Science Journal, 3, 28-34.
[19] De Martino et al., Enhanced culture of Borrelia garinii and Borrelia afzelii strains on a solid BSK-based medium in anaerobic conditions. Res. Microbiol. 2006, 157, 726-729.
[20] Laemmli, U. K. (1970) “Cleavage of structural proteins during the assembly of the head of bacteriophage T4”. Nature 227, 680-685.
[21] Candiano et al. Blue silver: a very sensitive colloidal Coomassie G-250 staining for proteome analysis. Electrophoresis 2004, 25, 1327-1333.
[22] Villiers, C et al. From secretome analysis to immunology: chitosan induces major alterations in the activation of dendritic cells via a TLR4-dependent mechanism. Mol. Cell. Proteomics MCP 2009, 8, 1252-1264.
[23] Perkins, D. N et al. Probability-based protein identification by searching sequence databases using mass spectrometry data. Electrophoresis 1999, 20, 3551-3567.
[24] Geer, L. Y. et al., Open mass spectrometry search algorithm. J. Proteome Res. 2004, 3, 958-964.
[25] Pham, T. V. et al. On the beta-binomial model for analysis of spectral count data in label-free tandem mass spectrometry-based proteomics. Bioinforma. Oxf. Engl. 2010, 26, 363-369.
[26] Balgley, B. M., et al. Comparative evaluation of tandem MS search algorithms using a target-decoy search strategy. Mol. Cell. Proteomics MCP 2007, 6, 1599-1608.
[27] Altschul, S. F. et al. Basic local alignment search tool. J. Mol. Biol. 1990, 215, 403-410.
[28] Berthold, S. W. et al. 1993. Chronic Lyme borreliosis in the laboratory mouse. Am. J. Pathol. 143:959-971.
[29] Kern A, et al. Tick saliva represses innate immunity and cutaneous inflammation in a murine model of Lyme disease. Vector Borne Zoonotic Dis. 2011 October; 11(10): 1343-50.
[30] Woods, A et al. 2008. MyD88 negatively controls hypergammaglobulinemia with autoantibody production during bacterial infection. Infect. Immun. 76: 1657-1667.
[31] Ouyang Z et al. 2012. Activation of the RpoN-RpoS regulatory pathway during the enzootic life cycle of Borrelia burgdorferi. BMC Microbiol. 12:44.
[32] Brisson D et al. Biodiversity of Borrelia burgdorferi strains in tissues of Lyme disease patients. PLoS One. 2011; 6(8):e22926.
[33] Steere A C et al. Vaccination against Lyme disease with recombinant Borrelia burgdorferi outer-surface lipoprotein A with adjuvant. Lyme Disease Vaccine Study Group. N Engl J Med. 1998 Jul. 23; 339(4):209-15.
[34] Embers M E, Narasimhan S. Vaccination against Lyme disease: past, present, and future. Front Cell Infect Microbiol. 2013; 3:6.
[35] Sterre et al. 1983. The spirochetal etiology of Lyme disease. N. Engl. J. Med. 308:733-740.

1. A vaccine composition comprising:
at least one polypeptide of Borrelia burgdorferi ss chosen from SEQ ID NOs: 10, 13, 2, 8, 9, 19, 25, 29, 33, 35, 43, 45 and 85.

2. The vaccine composition as claimed in claim 1, wherein the at least one polypeptide of Borrelia burgdorferi ss is chosen from SEQ ID NO: 10.

3. The vaccine composition as claimed in claim 1, comprising, in addition to the at least one polypeptide of claim 1, at least one polypeptide of Borrelia burgdorferi ss, Borrelia afzelii or Borrelia garinii chosen from SEQ ID NOs: 1, 3 to 7, 11, 12, 14 to 18, 20 to 24, 26 to 28, 30 to 32, 34, 36 to 42, 44, 46 to 84 and 86 to 92.

4. The composition as claimed in claim 1, comprising, in addition to the at least one polypeptide of claim 1, at least one other polypeptide of Borrelia burgdorferi ss, Borrelia afzelii or Borrelia garinii chosen from the groups (c1), (c2) and (c3),
said group (c1) comprising SEQ ID NOs: 19 to 28,
said group (c2) comprising SEQ ID NOs: 29 to 46 and 73 to 86,
said group (c3) comprising SEQ ID NOs: 47 to 72 and 87 to 92,
on the condition that, if said at least one polypeptide of claim 1:
is SEQ ID NO: 19 or 25, said at least one other polypeptide is included in the group (c2) or (c3), or
is SEQ ID NO: 29, 33, 35, 43, 45 or 85, said at least one other polypeptide is included in the group (c1) or (c3), or
is SEQ ID NO: 2, 8 or 9, said at least one other polypeptide is included in the group (c3), or
is SEQ ID NO: 10 or 13, said at least one other polypeptide is included in any of the groups (c1), (c2) and (c3).

5. The composition as claimed in claim 1, further comprising a pharmaceutically acceptable carrier.

6. The vaccine composition as claimed in claim 1, comprising an adjuvant.

7. The vaccine composition as claimed in claim 1, comprising at least one other active ingredient.

8. The vaccine composition as claimed in claim 1, for use as a medicament.

9. The vaccine composition as claimed in claim 1, for use in the prevention of Lyme disease.
